SEQUENCE LISTING (27 sequences)

SEQ ID NO: 1 - DNA, 4471 bp, Artificial Sequence
  lentiviral vector (RRL.PPT.CAGs.dTomato.pre) (nucleic acid sequence)
  promoter 1..232: /function=“RSV promoter”
  5′UTR 233..414: /note=“R/U5”
  5′UTR 415..432: /note=“Primer bindings site (PBS)”
  5′UTR 569..933: /note=“dGag”
  5′UTR 1079..1320: /note=“Rev-responsive element (RRE)”
  5′UTR 1804..1927: /note=“polypurine tract (PPT)”
  enhancer 1959..2338: /note=“Human Cytomegalovirus Immediate-Early (CMVie)”
  promoter 1962..2817: /note=“CAGs”
  promoter 2340..2617: /note=“Chicken beta-Actin promoter”
  gene 2843..3547: /note=“Transgene dTomato”
  3′UTR 3548..4156: /note=“Postranscriptional Regulatory Element (PRE)”
  LTR 4238..4471: /note=“self-inactivating (SIN) LTR”
ttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagctagcccgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggaggaccggtgaattcggatccgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtaaagcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 2 - DNA, 1536 bp, Artificial Sequence
  Vesicular stomatitis virus (VSV)-G ts (nucleic acid sequence)
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaaatggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccgcagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

SEQ ID NO: 3 - protein, 511 aa, Artificial Sequence
  Vesicular stomatitis virus (VSV)-G ts (amino acid sequence)
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYENGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTAERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 4 - DNA, 1539 bp, Cocal virus
  Cocal virus (COCV)-G (nucleic acid sequence)
atgaatttcctactcttgacatttattgtgttgccgttgtgcagccacgccaagttctccattgtattccctcaaagccaaaaaggcaattggaagaatgtaccatcatcttaccattactgcccttcaagttcggatcaaaactggcacaatgatttgcttggaatcacaatgaaagtcaaaatgcccaaaacacacaaagctattcaagcagacgggtggatgtgtcatgctgccaaatggatcactacctgtgactttcgctggtacggacccaaatacatcactcactccattcattccatccagcctacttcagagcagtgtaaagaaagcatcaagcaaacaaaacaaggtacttggatgagtcctggcttccctccacagaactgcgggtatgcaacagtaacagactctgtcgctgttgtcgtccaagccactcctcatcatgtcttggttgatgaatatactggagaatggatcgactctcaattccccaacgggaaatgtgaaaccgaagagtgcgagaccgtccacaactctaccgtatggtactctgactacaaagtaactggattatgtgacgcaactctggtagacacagagatcaccttcttctctgaagatggcaaaaaagaatctatcgggaagcccaacacaggctataggagcaactacttcgcttatgagaaaggggacaaagtatgtaaaatgaactactgcaagcatgcgggtgtgaggttgccttccggggtttggtttgagtttgtggatcaggatgtctacgccgccgccaaacttccagaatgccccgttggtgccactatctccgctccgacacagacctctgttgacgtaagtctcattctagatgtagagagaattttagattactctctgtgtcaagagacatggagcaagatccggtccaaacagccagtatcccctgttgaccttagttacttggcccccaagaatcctgggaccggaccggcattcacaatcatcaatggcactctgaagtactttgagaccagatacattcggattgatatagacaatccaatcatctccaagatggtggggaaaataagtggcagtcaaacagaacgagaattgtggacagagtggttcccctacgagggtgtcgagatagggccaaatgggattctcaaaacccctacaggatacaaattcccactcttcatgataggacacgggatgctagattccgacttgcacaagacgtcccaagcagaggtctttgaacatcctcaccttgcagaagcaccaaagcagttgccggaggaggagactttattttttggtgacacaggaatctccaaaaatccggtcgaactgattgaagggtggtttagtagttggaagagcactgtagtcacctttttctttgccataggagtatttatactactgtatgtagtggccagaattgtgatcgcagtgagatacagatatcaaggctcaaataacaaaagaatttacaatgatattgagatgagcagatttagaaaatga

SEQ ID NO: 5 - protein, 512 aa, Cocal virus
  Cocal virus (COCV)-G (amino acid sequence)
MNFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 6 - DNA, 1539 bp, Maraba virus
  Maraba virus (MARAV)-G (nucleic acid sequence)
atgttgagactttttctcttttgtttcttggccttaggagcccactccaaatttactatagtattccctcatcatcaaaaagggaattggaagaatgtgccttccacatatcattattgcccttctagttctgaccagaattggcataatgatttgactggagttagtcttcatgtgaaaattcccaaaagtcacaaagctatacaagcagatggctggatgtgccacgctgctaaatgggtgactacttgtgacttcagatggtacggacccaaatacatcacgcattccatacactctatgtcacccaccctagaacagtgcaagaccagtattgagcagacaaagcaaggagtttggattaatccaggctttccccctcaaagctgcggatatgctacagtgacggatgcagaggtggttgttgtacaagcaacacctcatcatgtgttggttgatgagtacacaggagaatggattgactcacaattggtggggggcaaatgttccaaggaggtttgtcaaacggttcacaactcgaccgtgtggcatgctgattacaagattacagggctgtgcgagtcaaatctggcatcagtggatatcaccttcttctctgaggatggtcaaaagacgtctttgggaaaaccgaacactggattcaggagtaatcactttgcttacgaaagtggagagaaggcatgccgtatgcagtactgcacacaatgggggatccgactaccttctggagtatggtttgaattagtggacaaagatctcttccaggcggcaaaattgcctgaatgtcctagaggatccagtatctcagctccttctcagacttctgtggatgttagtttgatacaagacgtagagaggatcttagattactctctatgccaggagacgtggagtaagatacgagccaagcttcctgtatctccagtagatctgagttatctcgccccaaaaaatccagggagcggaccggccttcactatcattaatggcactttgaaatatttcgaaacaagatacatcagagttgacataagtaatcccatcatccctcacatggtgggaacaatgagtggaaccacgactgagcgtgaattgtggaatgattggtatccatatgaagacgtagagattggtccaaatggggtgttgaaaactcccactggtttcaagtttccgctgtacatgattgggcacggaatgttggattccgatctccacaaatcctcccaggctcaagtcttcgaacatccacacgcaaaggacgctgcatcacagcttcctgatgatgagactttattttttggtgacacaggactatcaaaaaacccagtagagttagtagaaggctggttcagtagctggaagagcacattggcatcgttctttctgattataggcttgggggttgcattaatcttcatcattcgaattattgttgcgattcgctataaatacaaggggaggaagacccaaaaaatttacaatgatgtcgagatgagtcgattgggaaataaataa

SEQ ID NO: 7 - protein, 512 aa, Maraba virus
  Maraba virus (MARAV)-G (amino acid sequence)
MLRLFLFCFLALGAHSKFTIVFPHHQKGNWKNVPSTYHYCPSSSDQNWHNDLTGVSLHVKIPKSHKAIQADGWMCHAAKWVTTCDFRWYGPKYITHSIHSMSPTLEQCKTSIEQTKQGVWINPGFPPQSCGYATVTDAEVVVVQATPHHVLVDEYTGEWIDSQLVGGKCSKEVCQTVHNSTVWHADYKITGLCESNLASVDITFFSEDGQKTSLGKPNTGFRSNHFAYESGEKACRMQYCTQWGIRLPSGVWFELVDKDLFQAAKLPECPRGSSISAPSQTSVDVSLIQDVERILDYSLCQETWSKIRAKLPVSPVDLSYLAPKNPGSGPAFTIINGTLKYFETRYIRVDISNPIIPHMVGTMSGTTTERELWNDWYPYEDVEIGPNGVLKTPTGFKFPLYMIGHGMLDSDLHKSSQAQVFEHPHAKDAASQLPDDETLFFGDTGLSKNPVELVEGWFSSWKSTLASFFLIIGLGVALIFIIRIIVAIRYKYKGRKTQKIYNDVEMSRLGNK

SEQ ID NO: 8 - DNA, 1698 bp, RD114 retrovirus
  RD114 glycoprotein (nucleic acid sequence)
atgaaactcccaacaggaatggtcattttatgtagcctaataatagttcgggcagggtttgacgacccccgcaaggctatcgcattagtacaaaaacaacatggtaaaccatgcgaatgcagcggagggcaggtatccgaggccccaccgaactccatccaacaggtaacttgcccaggcaagacggcctacttaatgaccaaccaaaaatggaaatgcagagtcactccaaaaaatctcacccctagcgggggagaactccagaactgcccctgtaacactttccaggactcgatgcacagttcttgttatactgaataccggcaatgcagggcgaataataagacatactacacggccaccttgcttaaaatacggtctgggagcctcaacgaggtacagatattacaaaaccccaatcagctcctacagtccccttgtaggggctctataaatcagcccgtttgctggagtgccacagcccccatccatatctccgatggtggaggacccctcgatactaagagagtgtggacagtccaaaaaaggctagaacaaattcataaggctatgcatcctgaacttcaataccaccccttagccctgcccaaagtcagagatgaccttagccttgatgcacggacttttgatatcctgaataccacttttaggttactccagatgtccaattttagccttgcccaagattgttggctctgtttaaaactaggtacccctacccctcttgcgatacccactccctctttaacctactccctagcagactccctagcgaatgcctcctgtcagattatacctcccctcttggttcaaccgatgcagttctccaactcgtcctgtttatcttcccctttcattaacgatacggaacaaatagacttaggtgcagtcacctttactaactgcacctctgtagccaatgtcagtagtcctttatgtgccctaaacgggtcagtcttcctctgtggaaataacatggcatacacctatttaccccaaaactggacaggactttgcgtccaagcctccctcctccccgacattgacatcatcccgggggatgagccagtccccattcctgccattgatcattatatacatagacctaaacgagctgtacagttcatccctttactagctggactgggaatcaccgcagcattcaccaccggagctacaggcctaggtgtctccgtcacccagtatacaaaattatcccatcagttaatatctgatgtccaagtcttatccggtaccatacaagatttacaagaccaggtagactcgttagctgaagtagttctccaaaataggaggggactggacctactaacggcagaacaaggaggaatttgtttagccttacaagaaaaatgctgtttttatgctaacaagtcaggaattgtgagaaacaaaataagaaccctacaagaagaattacaaaaacgcagggaaagcctggcatccaaccctctctggaccgggctgcagggctttcttccgtacctcctacctctcctgggacccctactcaccctcctactcatactaaccattgggccatgcgttttcagtcgcctcatggccttcattaatgatagacttaatgttgtacatgccatggtgctggcccagcaataccaagcactcaaagctgaggaagaagctcaggattga

SEQ ID NO: 9 - protein, 565 aa, RD114 retrovirus
  RD114 glycoprotein (amino acid sequence)
MKLPTGMVILCSLIIVRAGFDDPRKAIALVQKQHGKPCECSGGQVSEAPPNSIQQVTCPGKTAYLMTNQKWKCRVTPKNLTPSGGELQNCPCNTFQDSMHSSCYTEYRQCRANNKTYYTATLLKIRSGSLNEVQILQNPNQLLQSPCRGSINQPVCWSATAPIHISDGGGPLDTKRVWTVQKRLEQIHKAMHPELQYHPLALPKVRDDLSLDARTFDILNTTFRLLQMSNFSLAQDCWLCLKLGTPTPLAIPTPSLTYSLADSLANASCQIIPPLLVQPMQFSNSSCLSSPFINDTEQIDLGAVTFTNCTSVANVSSPLCALNGSVFLCGNNMAYTYLPQNWTGLCVQASLLPDIDIIPGDEPVPIPAIDHYIHRPKRAVQFIPLLAGLGITAAFTTGATGLGVSVTQYTKLSHQLISDVQVLSGTIQDLQDQVDSLAEVVLQNRRGLDLLTAEQGGICLALQEKCCFYANKSGIVRNKIRTLQEELQKRRESLASNPLWTGLQGFLPYLLPLLGPLLTLLLILTIGPCVFSRLMAFINDRLNVVHAMVLAQQYQALKAEEEAQD

SEQ ID NO: 10 - DNA, 1692 bp, Baboon endogenous virus strain M7
  Baboon endogenous virus strain M7 (BaEV)-GP (nucleic acid sequence)
atgggattcacaacaaagataatcttcttatacaacctagtactggtctacgcggggtttgacgaccctcgcaaagccatagaactagtacaaaagcgatatggccgaccatgcgattgcagcggaggacaagtgtccgagcccccgtcagacagggtcagtcaagtgacttgctcaggcaagacagcttacttaatgcccgaccaaagatggaaatgtaagtcaattccaaaagacacctccccaagcgggccactccaagagtgcccctgtaattcttaccagtcctcagtacacagttcttgttatacctcataccaacaatgcagatcaggcaataagacatattatacggctactctgctaaaaacacaaactgggggcaccagtgatgtacaagtattaggatccaccaacaaacttatacaatctccctgtaatggcataaaagggcagtctatttgctggagcactacagctcctatccacgtctctgatggaggaggtccattagacaccacaagaattaaaagtgttcagagaaaactggaagaaattcataaagccctatatcctgaacttcagtatcaccctttggccatacctaaggttagagataacctcatggtcgatgcccagactttaaacattctcaatgccacttacaacttactcctaatgtccaacacgagcctagtggacgactgttggctttgtttaaaattaggtccccctactcccctcgcaatacctaacttcctattatcctacgtgactcgctcctcggataatatctcttgtttaataattcccccccttctagttcaaccgatgcagttttccaattcatcttgcctcttttccccctcctacaacagtacagaagaaatagatctaggccatgttgccttcagcaactgtacctccataaccaatgtcaccggtcccatatgcgctgtaaatggttcggtctttctctgtggcaataacatggcatacacttatctacccacgaactggacggggctttgcgtcctagcaactctcctccccgacattgacatcattcccggagatgaaccggtccccatccctgctattgatcattttatatatagacctaaacgggccatacagtttattcctttactagcagggctagggatcaccgcagccttcacaacaggagctacaggcctaggtgtctctgtgacccaatatacaaaattatctaatcagctaatttctgatgtacaaatcttatctagcaccatacaagatctgcaagatcaagtagactcattagccgaagtggttctccagaacagaagggggctagatctacttacagcagaacaaggaggaatctgtttagccctgcaagaaaaatgctgcttttatgttaacaagtcagggattgtgagagacaaaataaaaaccttacaagaagaactagaaagacgtagaaaagatctagcttccaacccactttggactgggcttcaagggctcctcccttacctcctgccctttcttggccctctacttaccctcctgctcttactcaccattgggccgtgcatttttaaccgtctaaccgcttttattaatgataagttaaacataatacacgctatggtgctaacccaacagtatcaggtgctcagaaccgatgaagaagctcaagattga

SEQ ID NO: 11 - protein, 563 aa, Baboon endogenous virus strain M7
  Baboon endogenous virus strain M7 (BaEV)-GP (amino acid sequence)
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLRTDEEAQD

SEQ ID NO: 12 - DNA, 2058 bp, Gibbon ape leukemia virus
  Gibbon ape leukemia virus strain SEATO (GALV)-GP (nucleic acid sequence)
atggtattgctgcctgggtccatgcttctcacctcaaacctgcaccaccttcggcaccagatgagtcctgggagctggaaaagactgatcatcctcttaagctgcgtattcggcggcggcgggacgagtctgcaaaataagaacccccaccagcccatgaccctcacttggcaggtactgtcccaaactggagacgttgtctgggatacaaaggcagtccagcccccttggacttggtggcccacacttaaacctgatgtatgtgccttggcggctagtcttgagtcctgggatatcccgggaaccgatgtctcgtcctctaaacgagtcagacctccggactcagactatactgccgcttataagcaaatcacctggggagccatagggtgcagctaccctcgggctaggactagaatggcaagctctaccttctacgtatgtccccgggatggccggaccctttcagaagctagaaggtgcggggggctagaatccctatactgtaaagaatgggattgtgagaccacggggaccggttattggctatctaaatcctcaaaagacctcataactgtaaaatgggaccaaaatagcgaatggactcaaaaatttcaacagtgtcaccagaccggctggtgtaacccccttaaaatagatttcacagacaaaggaaaattatccaaggactggataacgggaaaaacctggggattaagattctatgtgtctggacatccaggcgtacagttcaccattcgcttaaaaatcaccaacatgccagctgtggcagtaggtcctgacctcgtccttgtggaacaaggacctcctagaacgtccctcgctctcccacctcctcttcccccaagggaagcgccaccgccatctctccccgactctaactccacagccctggcgactagtgcacaaactcccacggtgagaaaaacaattgttaccctaaacactccgcctcccaccacaggcgacagactttttgatcttgtgcagggggccttcctaaccttaaatgctaccaacccaggggccactgagtcttgctggctttgtttggccatgggccccccttattatgaagcaatagcctcatcaggagaggtcgcctactccaccgaccttgaccggtgccgctgggggacccaaggaaagctcaccctcactgaggtctcaggacacgggttgtgcataggaaaggtgccctttacccatcagcatctctgcaatcagaccctatccatcaattcctccggagaccatcagtatctgctcccctccaaccatagctggtgggcttgcagcactggcctcaccccttgcctctccacctcagtttttaatcagactagagatttctgtatccaggtccagctgattcctcgcatctattactatcctgaagaagttttgttacaggcctatgacaattctcaccccaggactaaaagagaggctgtctcacttaccctagctgttttactggggttgggaatcacggcgggaataggtactggttcaactgccttaattaaaggacctatagacctccagcaaggcctgacaagcctccagatcgccatagatgctgacctccgggccctccaagactcagtcagcaagttagaggactcactgacttccctgtccgaggtagtgctccaaaataggagaggccttgacttgctgtttctaaaagaaggtggcctctgtgcggccctaaaggaagagtgctgtttttacatagaccactcaggtgcagtacgggactccatgaaaaaactcaaagaaaaactggataaaagacagttagagcgccagaaaagccaaaactggtatgaaggatggttcaataactccccttggttcactaccctgctatcaaccatcgctgggcccctattactcctccttctgttgctcatcctcgggccatgcatcatcaataagttagttcaattcatcaatgataggataagtgcagttaaaattctggtccttagacaaaaatatcaggccctagagaacgaaggtaacctttaa

SEQ ID NO: 13 - protein, 685 aa, Gibbon ape leukemia virus
  Gibbon ape leukemia virus strain SEATO (GALV)-GP (amino acid sequence)
MVLLPGSMLLTSNLHHLRHQMSPGSWKRLIILLSCVFGGGGTSLQNKNPHQPMTLTWQVLSQTGDVVWDTKAVQPPWTWWPTLKPDVCALAASLESWDIPGTDVSSSKRVRPPDSDYTAAYKQITWGAIGCSYPRARTRMASSTFYVCPRDGRTLSEARRCGGLESLYCKEWDCETTGTGYWLSKSSKDLITVKWDQNSEWTQKFQQCHQTGWCNPLKIDFTDKGKLSKDWITGKTWGLRFYVSGHPGVQFTIRLKITNMPAVAVGPDLVLVEQGPPRTSLALPPPLPPREAPPPSLPDSNSTALATSAQTPTVRKTIVTLNTPPPTTGDRLFDLVQGAFLTLNATNPGATESCWLCLAMGPPYYEAIASSGEVAYSTDLDRCRWGTQGKLTLTEVSGHGLCIGKVPFTHQHLCNQTLSINSSGDHQYLLPSNHSWWACSTGLTPCLSTSVFNQTRDFCIQVQLIPRIYYYPEEVLLQAYDNSHPRTKREAVSLTLAVLLGLGITAGIGTGSTALIKGPIDLQQGLTSLQIAIDADLRALQDSVSKLEDSLTSLSEVVLQNRRGLDLLFLKEGGLCAALKEECCFYIDHSGAVRDSMKKLKEKLDKRQLERQKSQNWYEGWFNNSPWFTTLLSTIAGPLLLLLLLLILGPCIINKLVQFINDRISAVKILVLRQKYQALENEGNL

SEQ ID NO: 14 - DNA, 1965 bp, Amphotropic murine leukemia virus
  4070A Amphotropic Murine leukemia virus (Ampho)-GP (nucleic acid sequence)
atggcgcgttcaacgctctcaaaaccccctcaagataagattaacccgtggaagcccttaatagtcatgggagtcctgttaggagtagggatggcagagagcccccatcaggtctttaatgtaacctggagagtcaccaacctgatgactgggcgtaccgccaatgccacctccctcctgggaactgtacaagatgccttcccaaaattatattttgatctatgtgatctggtcggagaggagtgggacccttcagaccaggaaccgtatgtcgggtatggctgcaagtaccccgcagggagacagcggacccggacttttgacttttacgtgtgccctgggcataccgtaaagtcggggtgtgggggaccaggagagggctactgtggtaaatgggggtgtgaaaccaccggacaggcttactggaagcccacatcatcgtgggacctaatctcccttaagcgcggtaacaccccctgggacacgggatgctctaaagttgcctgtggcccctgctacgacctctccaaagtatccaattccttccaaggggctactcgagggggcagatgcaaccctctagtcctagaattcactgatgcaggaaaaaaggctaactgggacgggcccaaatcgtggggactgagactgtaccggacaggaacagatcctattaccatgttctccctgacccggcaggtccttaatgtgggaccccgagtccccatagggcccaacccagtattacccgaccaaagactcccttcctcaccaatagagattgtaccggctccacagccacctagccccctcaataccagttaccccccttccactaccagtacaccctcaacctcccctacaagtccaagtgtcccacagccacccccaggaactggagatagactactagctctagtcaaaggagcctatcaggcgcttaacctcaccaatcccgacaagacccaagaatgttggctgtgcttagtgtcgggacctccttattacgaaggagtagcggtcgtgggcacttataccaatcattccaccgctccggccaactgtacggccacttcccaacataagcttaccctatctgaagtgacaggacagggcctatgcatgggggcagtacctaaaactcaccaggccttatgtaacaccacccaaagcgccggctcaggctcctactaccttgcagcacccgccggaacaatgtgggcttgcagcactggattgactccctgcttgtccaccacggtgctcaatctaaccacagattattgtgtattagttgaactctggcccagagtaatttaccactcccccgattatatgtatggtcagcttgaacagcgtaccaaatataaaagagagccagtatcattgaccctggcccttctactaggaggattaaccatgggagggattgcagctggaatagggacggggaccactgccttaattaaaacccagcagtttgagcagcttcatgccgctatccagacagacctcaacgaagtcgaaaagtcaattaccaacctagaaaagtcactgacctcgttgtctgaagtagtcctacagaaccgcagaggcctagatttgctattcctaaaggagggaggtctctgcgcagccctaaaagaagaatgttgtttttatgcagaccacacggggctagtgagagacagcatggccaaattaagagaaaggcttaatcagagacaaaaactatttgagacaggccaaggatggttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccatga

SEQ ID NO: 15 - protein, 654 aa, Amphotropic murine leukemia virus
  4070A Amphotropic Murine leukemia virus (Ampho)-GP (amino acid sequence)
MARSTLSKPPQDKINPWKPLIVMGVLLGVGMAESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQALVLTQQYHQLKPIEYEP

SEQ ID NO: 16 - DNA, 1938 bp, Murine leukemia virus
  10A1 Murine leukemia virus (MLV)-GP (nucleic acid sequence)
atggaaggtccagcgttctcaaaaccccttaaagataagattaacccgtggaagtccttaatggtcatgggggtctatttaagagtagggatggcagagagcccccatcaggtctttaatgtaacctggagagtcaccaacctgatgactgggcgtaccgccaatgccacctcccttttaggaactgtacaagatgccttcccaagattatattttgatctatgtgatctggtcggagaagagtgggacccttcagaccaggaaccatatgtcgggtatggctgcaaataccccggagggagaaagcggacccggacttttgacttttacgtgtgccctgggcataccgtaaaatcggggtgtggggggccaagagagggctactgtggtgaatggggttgtgaaaccaccggacaggcttactggaagcccacatcatcatgggacctaatctcccttaagcgcggtaacaccccctgggacacgggatgctccaaaatggcttgtggcccctgctacgacctctccaaagtatccaattccttccaaggggctactcgagggggcagatgcaaccctctagtcctagaattcactgatgcaggaaaaaaggctaattgggacgggcccaaatcgtggggactgagactgtaccggacaggaacagatcctattaccatgttctccctgacccgccaggtcctcaatatagggccccgcatccccattgggcctaatcccgtgatcactggtcaactacccccctcccgacccgtgcagatcaggctccccaggcctcctcagcctcctcctacaggcgcagcctctatagtccctgagactgccccaccttctcaacaacctgggacgggagacaggctgctaaacctggtagaaggagcctatcaggcgcttaacctcaccaatcccgacaagacccaagaatgttggctgtgcttagtgtcgggacctccttattacgaaggagtagcggtcgtgggcacttataccaatcattctaccgccccggccagctgtacggccacttcccaacataagcttaccctatctgaagtgacaggacagggcctatgcatgggagcactacctaaaactcaccaggccttatgtaacaccacccaaagtgccggctcaggatcctactaccttgcagcacccgctggaacaatgtgggcttgtagcactggattgactccctgcttgtccaccacgatgctcaatctaaccacagactattgtgtattagttgagctctggcccagaataatttaccactcccccgattatatgtatggtcagcttgaacagcgtaccaaatataagagggagccagtatcgttgaccctggcccttctgctaggaggattaaccatgggagggattgcagctggaatagggacggggaccactgccctaatcaaaacccagcagtttgagcagcttcacgccgctatccagacagacctcaacgaagtcgaaaaatcaattaccaacctagaaaagtcactgacctcgttgtctgaagtagtcctacagaaccgaagaggcctagatttgctcttcctaaaagagggaggtctctgcgcagccctaaaagaagaatgttgtttttatgcagaccacacgggactagtgagagacagcatggccaaactaagggaaaggcttaatcagagacaaaaactatttgagtcaggccaaggttggttcgaagggcagtttaatagatccccctggtttaccaccttaatctccaccatcatgggacctctaatagtactcttactgatcttactctttggaccctgcattctcaatcgattggtccaatttgttaaagacaggatctcagtggtccaggctctggttttgactcaacaatatcaccagctaaaacctatagagtacgagccatga

SEQ ID NO: 17 - protein, 645 aa, Murine leukemia virus
  10A1 Murine leukemia virus (MLV)-GP (amino acid sequence)
MEGPAFSKPLKDKINPWKSLMVMGVYLRVGMAESPHQVFNVTWRVTNLMTGRTANATSLLGTVQDAFPRLYFDLCDLVGEEWDPSDQEPYVGYGCKYPGGRKRTRTFDFYVCPGHTVKSGCGGPREGYCGEWGCETTGQAYWKPTSSWDLISLKRGNTPWDTGCSKMACGPCYDLSKVSNSFQGATRGGRCNPLVLEFTDAGKKANWDGPKSWGLRLYRTGTDPITMFSLTRQVLNIGPRIPIGPNPVITGQLPPSRPVQIRLPRPPQPPPTGAASIVPETAPPSQQPGTGDRLLNLVEGAYQALNLTNPDKTQECWLCLVSGPPYYEGVAVVGTYTNHSTAPASCTATSQHKLTLSEVTGQGLCMGALPKTHQALCNTTQSAGSGSYYLAAPAGTMWACSTGLTPCLSTTMLNLTTDYCVLVELWPRIIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFESGQGWFEGQFNRSPWFTTLISTIMGPLIVLLLILLFGPCILNRLVQFVKDRISVVQALVLTQQYHQLKPIEYEP

SEQ ID NO: 18 - DNA, 1590 bp, Piry virus
  Piry virus (PIRYV)-G (nucleic acid sequence)
atggatctctttcccattttggtcgtggtgctcatgacagatactgtcttagggaagtttcaaattgtcttcccggatcagaatgaactggagtggagaccagttgtgggtgactctcggcattgcccacagtcatcagaaatgcaattcgatggaagcagatcccagaccatactgactgggaaagctcccgtggggatcacgccctctaaatcagatggatttatctgccatgccgcaaaatgggtgacaacatgtgatttcaggtggtatgggccgaaatacatcactcattcaatacatcatctgagaccgacaacatcagactgtgagacagctctccaaaggtataaagatgggagcttaatcaatcttggattccccccagaatcctgcggttatgcaacagtcacagattctgaggcaatgttggtccaagtgactccccaccacgttggggtggatgattatagaggtcactggatcgacccactatttccaggaggagaatgctccaccaatttttgtgatacagtccacaattcatcggtgtggatccccaagagtcaaaagactgacatctgtgcccagtctttcaaaaatatcaagatgaccgcatcttacccctcagaaggagcattggtgagtgacagatttgccttccacagtgcatatcatccaaatatgccggggtcaactgtttgcataatggacttttgcgaacaaaaggggttgagattcacaaatggagagtggatgggtctcaatgtggagcaatccatccgagagaagaagataagtgccatcttcccaaattgtgttgcagggactgaaatccgagccacactagaatcagaaggggcaagaactttgacgtgggagactcaaagaatgctagattactctttgtgtcagaacacctgggacaaagtttccaggaaagaacctctcagtccgcttgacttgagctatctgtcaccaagggctccagggaaaggcatggcctataccgtcataaacggaaccctgcattcggctcatgctaaatacattagaacctggattgattatggagaaatgaaggaaattaaaggtggacgtggagaatattccaaggctcctgagctcctctggtcccagtggttcgattttggaccgttcaaaattggaccgaatggactcctgcacacagggaaaacctttaaattccctctttatttgatcggagcaggcataattgacgaagatctgcatgaactagatgaggctgctcccattgatcacccacaaatgcctgacgcgaaaagcgttcttccagaagatgaagagatattcttcggagacacaggtgtatccaaaaaccctatcgagttgattcaaggatggttctcaaattggagagagagtgtaatggcaatagtcggaattgttctactcatcgttgtgacatttctggcgatcaagacggtccgggtgcttaattgtctctggagacccagaaagaaaagaatcgtcagacaagaagtagatgttgaatcccgactaaaccattttgagatgagaggctttcctgaatatgttaagagataa

SEQ ID NO: 19 - protein, 529 aa, Piry virus
  Piry virus (PIRYV)-G (amino acid sequence)
MDLFPILVVVLMTDTVLGKFQIVFPDQNELEWRPVVGDSRHCPQSSEMQFDGSRSQTILTGKAPVGITPSKSDGFICHAAKWVTTCDFRWYGPKYITHSIHHLRPTTSDCETALQRYKDGSLINLGFPPESCGYATVTDSEAMLVQVTPHHVGVDDYRGHWIDPLFPGGECSTNFCDTVHNSSVWIPKSQKTDICAQSFKNIKMTASYPSEGALVSDRFAFHSAYHPNMPGSTVCIMDFCEQKGLRFTNGEWMGLNVEQSIREKKISAIFPNCVAGTEIRATLESEGARTLTWETQRMLDYSLCQNTWDKVSRKEPLSPLDLSYLSPRAPGKGMAYTVINGTLHSAHAKYIRTWIDYGEMKEIKGGRGEYSKAPELLWSQWFDFGPFKIGPNGLLHTGKTFKFPLYLIGAGIIDEDLHELDEAAPIDHPQMPDAKSVLPEDEEIFFGDTGVSKNPIELIQGWFSNWRESVMAIVGIVLLIVVTFLAIKTVRVLNCLWRPRKKRIVRQEVDVESRLNHFEMRGFPEYVKR

SEQ ID NO: 20 - DNA, 1536 bp, Vesicular stomatitis virus
  Vesicular stomatitis virus (VSV)-G serotype Indiana (nucleic acid sequence)
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

SEQ ID NO: 21 - protein, 511 aa, Vesicular stomatitis virus
  Vesicular stomatitis virus (VSV)-G serotype Indiana (amino acid sequence)
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 22 - DNA, 1554 bp, Vesicular stomatitis virus
  Vesicular stomatitis virus (VSV)-G serotype New Jersey (nucleic acid sequence)
atgttgtcttatctaatctttgcacttgccgtttcgcccattttgggcaaaattgaaattgtgtttcctcaacataccactggggattggaagagagttccccatgaatataattattgccctaccagcgcagacaagaactcacatgggactcaaacaggaatccctgttgagttaacaatgccaaaaggactaacaacccatcaagttgaaggatttatgtgtcactcagccttgtggatgaccacttgtgacttcagatggtatgggccaaaatacataacccattccatacataatgaagagcctacagattatcaatgtttggaggccattaagtcatacaaagatggagtcagtttcaatccagggtttcctcctcagagctgcgggtatggcacagttaccgatgccgaagcccatattgtgacagttactccccactctgtcaaagtggacgagtacacgggggaatggatcgatccacatttcatcggaggaaggtgcaaaggacaaatttgtgaaacagtccataattccacaaaatggtttacgtcctctgatggagaaagtgtctgcagtcaattgtttactttggttggaggaatttttttctctgattcagaagagattacctccatggggttaccagaaacaggaatcagaagtaattacttcccctacatatctacagagggaatttgcaaaatgccgttttgcagaaaacaggggtacaagcttaaaaatgacctctggttccagatcatggacccagacctggataaaacggttagagatctccctcatattaaggactgtgacctctcctcgtccataatcacaccaggagaacatgctacagacatctcactgatatcagatgttgaaaggatcctggactatgctctttgtcagaatacatggagtaaaattgaatcgggagaaccaattactccggtagatctcagctatcttgggccaaaaaacccaggggttgggccggtcttcaccatcattaacggttccctgcattattttacatcgaagtatctgcgagtcgaattagaaagtcctgtcatacccagaatggaaggaaaagttgcaggaactaggattgtacggcaattgtgggatcagtggtttcctttcggagaagttgagattggacccaatggtgtgttgaaaacgaagcaagggtataaattcccactacacatcattggaactggagaagtagacagtgacatcaaaatggaaagggttgtcaagcactgggaacacccccatattgaggccgctcagacatttttaaaaaaagatgacacaggagaagtcctttattatggcgacaccggagtgtcgaaaaatccagttgaattagtcgagggatggtttagtggatggaggagctccctcatgggagtgctggctgtgattataggatttgtgattttaatgtttttaattaaattgattggagtcttatctagccttttcagacctaaacgcaggccaatctacaaatcagacgtggaaatggctcatttccgttaa

SEQ ID NO: 23 - protein, 517 aa, Vesicular stomatitis virus
  Vesicular stomatitis virus (VSV)-G serotype New Jersey (amino acid sequence)
MLSYLIFALAVSPILGKIEIVFPQHTTGDWKRVPHEYNYCPTSADKNSHGTQTGIPVELTMPKGLTTHQVEGFMCHSALWMTTCDFRWYGPKYITHSIHNEEPTDYQCLEAIKSYKDGVSFNPGFPPQSCGYGTVTDAEAHIVTVTPHSVKVDEYTGEWIDPHFIGGRCKGQICETVHNSTKWFTSSDGESVCSQLFTLVGGIFFSDSEEITSMGLPETGIRSNYFPYISTEGICKMPFCRKQGYKLKNDLWFQIMDPDLDKTVRDLPHIKDCDLSSSIITPGEHATDISLISDVERILDYALCQNTWSKIESGEPITPVDLSYLGPKNPGVGPVFTIINGSLHYFTSKYLRVELESPVIPRMEGKVAGTRIVRQLWDQWFPFGEVEIGPNGVLKTKQGYKFPLHIIGTGEVDSDIKMERVVKHWEHPHIEAAQTFLKKDDTGEVLYYGDTGVSKNPVELVEGWFSGWRSSLMGVLAVIIGFVILMFLIKLIGVLSSLFRPKRRPIYKSDVEMAHFR

SEQ ID NO: 24 - DNA, 7712 bp, Artificial Sequence
  Prototypical transfer vector plasmid encoding the lentiviral vector genome (pRRL.PPT.CAGs.dTomato.pre nosal) (nucleic acid sequence)
  promoter 4..235: /note=“RSV promoter”
  LTR 236..417: /note=“R/U5”
  5′UTR 418..435: /note=“Primer Binding Site (PBS)”
  5′UTR 572..936: /note=“dGag”
  5′UTR 1082..1323: /note=“Rev-responsive element (RRE)”
  5′UTR 1807..1930: /note=“Polypurine tract (PPT)”
  enhancer 1965..2341: /note=“Human Cytomegalovirus Immediate-Early (CMVie)”
  promoter 1965..2820: /note=“CAGs”
  promoter 2343..2620: /note=“Chicken beta-Actin promoter”
  gene 2846..3551: /note=“Transgene dTomato”
  3′UTR 3551..4159: /note=“Posttranscriptional Regulatory Element (PRE)”
  LTR 4241..4474: /note=“Self-inactivating (SIN) LTR”
  polyA_signal 4675..4881: /note=“SV40 pA/Ori”
  rep_origin 5042..5500: /note=“F1 ORI”
  gene 5632..6489: /note=“Ampicillin resistance gene”
agcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagctagcccgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggaggaccggtgaattcggatccgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtaaagcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgca

SEQ ID NO: 25 - DNA, 11035 bp, Artificial Sequence
  Packaging plasmid (pCDNA3.g/p.4xCTE) (nucleic acid sequence)
  misc_feature 2..769: /note=“4x Constitutive Transport Element (CTE)”
  polyA_signal 847..1071: /note=“bGH pA”
  promoter 5482..6069: /note=“CMV promoter”
  intron 6157..6700: /note=“Beta Globin Intron”
  gene 6722..11028: /note=“HIV-1 gag/pol”
aattcctgcagcccgggatccactagttctagaccacctcccctgtgagctagactggacagccaatgacgggtaagagagtgacatttctcactaacctaagacaggagggccgtcaaagctactgcctaatccaatgacgggtaatagtgacaagaaatgtatcactccaacctaagacaggcgcagcctccgagggatgtgttgatcagctagaccacctccctgtgagctagactggacagccaatgacgggtaagagagtgacatttctcactaacctaagacaggagggccgtcaaagctactgcctaatccaatgacgggtaatagtgacaagaaatgtatcactccaacctaagacaggcgcagcctccgagggatgtgttgatcagctagaccacctcccctgtgagctagactggacagccaatgacgggtaagagagtgacatttctcactaacctaagacaggagggccgtcaaagctactgcctaatccaatgacgggtaatagtgacaagaaatgtatcactccaacctaagacaggcgcagcctccgagggatgtgttgatcagctagaccacctcccctgtgagctagactggacagccaatgacgggtaagagagtgacatttctcactaacctaagacaggagggccgtcaaagctactgcctaatccaatgacgggtaatagtgacaagaaatgtatcactccaacctaagacaggcgcagcctccgagggatgtgttgatcagctagagcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcgagatctgccgccgccatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatgg

SEQ ID NO: 26 - DNA, 4180 bp, Artificial Sequence
  Rev-encoding plasmid (pRSV.Rev) (nucleic acid sequence)
  promoter 238..635: /note=“RSV promoter”
  gene 756..1106: /note=“Rev”
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagctcgagctcgtttagtgaaccgtcagatcgcctggagacgccatccacgctgttttgacctccatagaagacaccgggaccgatccagcctcccctcgaagctagtcgattaggcatctcctatggcaggaagaagcggagacagcgacgaagacctcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtgctgttagcttgctcaatgccacagctatagcagtagctgaggggacagatagggttatagaagtagtacaagaagcttggcactggccgtcgttttacaacgtcgtgatctgagcctgggagatctctggctaactagggaacccactgcttaagcctcaataaagcttgccttgagtgcttcaagtagtgtgtgcccgtctgttgtgtgactctggtaactagagatcaggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 27 - DNA, 4827 bp, Artificial Sequence
  Env-encoding plasmid (pHCMV.Env GP) without insert Env GP (nucleic acid sequence)
  promoter 1..752: /note=“CMV promoter”
  intron 760..1404: /note=“Globin intron”
  gene 1419..1420: /note=“Transgene/Env GP Insert Site”
  polyA_signal 1435..1957: /note=“Globin polyA”
  rep_origin 2398..2986: /note=“ORI”
  gene 3157..4017: /note=“Ampicillin resistance (AmpR)”
  promoter 4018..4122: /note=“AmpR promoter”
  rep_origin 4148..4603: /note=“F1 ORI”
gagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggtcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacagctcctgggcaacgtgctggttgttgtgctgtctcatcattttggcaaagaattcctcgacggatccgcctaaggatccgtcgaggaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacggatcggccgcaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctaga

(19) United States
(12) Patent Application Publication
(10) Pub. No.: US 2024/0299581 A1
(43) Pub. Date: Sep. 12, 2024

(54) LENTIVIRAL VECTOR TECHNOLOGY FOR INNER EAR GENE THERAPY

(71) Applicant: Medizinische Hochschule Hannover, Hannover (DE)

(72) Inventors: Axel SCHAMBACH, Hannover (DE); Michael MORGAN, Langenhagen (DE); Juliane SCHOTT, Hannover (DE); Hildegard BÜNING, Hannover (DE); Hinrich STAECKER, Leawood, KS (US); Athanasia WARNECKE, Burgwedel (DE)

(21) Appl. No.: 18/254,669

(22) PCT Filed: Dec. 3, 2021
(86) PCT No.: PCT/EP2021/084131

§ 371 Date: May 26, 2023

(30) Foreign Application Priority Data
Dec. 3, 2020 (EP) 20211616.6

(51) Int. Cl. A61K 48/00; A61K 9/00; A61P 27/16; C12N 7/00; C12N 15/86

(52) U.S. Cl. CPC A61K 48/005; A61K 9/0046; A61K 48/0075; A61P 27/16; C12N 7/00; C12N 15/86; C12N 2740/16022; C12N 2740/16043; C12N 2740/16051; C12N 2830/48

(57) ABSTRACT

The present invention relates to the field of gene therapy for the treatment of sensorineural hearing loss. In particular, the invention discloses a composition comprising a 3^{rd} generation lentiviral vector pseudotyped with a viral envelope glycoprotein capable of binding to a receptor expressed in a cell of the inner ear for use in the treatment or prevention of sensorineural hearing loss, wherein said composition has a viral titer of at least 10⁷ TU/mL and is administered to the inner ear of a subject suffering from or in danger to undergo sensorineural hearing loss. The viral envelope glycoprotein is capable of binding to a receptor selected from the group consisting of the LDL-receptor and LDL-R family members, the SLC1 A5-receptor, the Pit1/2-receptor and the PIRYV-G-receptor. Preferably, the viral glycoprotein is MARAV-G, COCV-G, VSV-G, VSV-G ts or PIRYV-G, most preferably, MARAV-G. The lentiviral vector further comprises a cargo sequence comprising, e.g. a protein-coding gene, a miRNA, an shRNA, a lncRNA or an sgRNA, wherein expression of said cargo sequence in the cell of the inner ear is capable of reducing, eliminating or preventing at least a symptom of sensorineural hearing loss in the subject. The invention further discloses a method for treating sensorineural hearing loss in a subject in need thereof.

The present invention relates to the field of gene therapy for the treatment of sensorineural hearing loss. In particular, the invention discloses a composition comprising a 3^{rd} generation lentiviral vector pseudotyped with a viral envelope glycoprotein capable of binding to a receptor expressed in a cell of the inner ear for use in the treatment or prevention of sensorineural hearing loss, wherein said composition has a viral titer of at least 10⁷ TU/mL and is administered to the inner ear of a subject suffering from or in danger to undergo sensorineural hearing loss. The viral envelope glycoprotein is capable of binding to a receptor selected from the group consisting of the LDL-receptor (LDL-R) and LDL-R family members, the SLC1A5-receptor, the Pit1/2-receptor and the PIRYV-G-receptor. Preferably, the viral glycoprotein is MARAV-G, COCV-G, VSV-G, VSV-G ts, or PIRYV-G. The lentiviral vector further comprises a cargo sequence comprising, e.g. a protein-coding gene, a miRNA, an shRNA, a lncRNA or an sgRNA, wherein expression of said cargo sequence in the cell of the inner ear is capable of reducing, eliminating or preventing at least a symptom of sensorineural hearing loss in the subject. The invention further discloses a method for treating sensorineural hearing loss in a subject in need thereof.

Approximately 466 million people suffer from disabling hearing loss, with substantial social and economic consequences (Blanc et al., 2020, rAAV-Mediated Cochlear Gene Therapy: Prospects and Challenges for Clinical Application. J. Clin Med. 9(2), 589). Sensorineural hearing loss (SNHL) is caused by dysfunction or degeneration of structures of the inner ear, including hair cells (HC), spiral ganglion neurons (SGN), supporting cells, the tectorial membrane and the stria vascularis. Especially HC (inner and outer HC, residing in the Organ of Corti) and SGN, both of which are part of the sensory epithelium of the cochlea, play important roles in the transduction of an acoustic stimulus from a mechanical vibration into neural impulses and the transmission of the latter to the primary auditory cortex in the brain.

Current treatment options for SNHL are limited to the use of medical devices. These are based on physico-mechanical amplification of sound (hearing aid) or direct stimulation of SGN (cochlear implant). While these treatment options provide benefit to a great proportion of patients, they are not applicable to all patients, as their use is restricted if cell types relevant for transmission of the signal to the brain are too damaged or degenerated. Furthermore, hearing aids and cochlear implants do not result in a hearing quality comparable to unaffected individuals, and the resolution achieved with a cochlear implant is often only suboptimal (Blanc et al., 2020). As a large percentage of SNHL cases are due to genetic aberrations, gene therapy to provide an intact copy of the defective gene in relevant cell types of the inner ear is a highly attractive alternative to current treatment options, or even the only treatment option in some cases. Moreover, genetic protection may be a suitable means to protect against or prevent the occurrence of acquired forms of SNHL, such as those caused by drug-induced, e.g., chemotherapeutic- or antibiotic-induced ototoxicity. Cell type-specific protein expression may be achieved by use of cell-specific promoters, specific viral subtypes, or a combination of both.

At present, vectors derived from adenovirus, adeno-associated virus (AAV), herpes simplex virus, vaccinia virus, helper-dependent virus and retrovirus have been tested for targeted gene delivery into the cochlea (Izumikawa et al., 2005, Auditory hair cell replacement and hearing improvement by Atoh1 gene therapy in deaf mammals. Nature Medicine 11(3), 271-276, Tao et al., 2018, Delivery of Adeno-Associated Virus Vectors in Adult Mammalian Inner-Ear Cell Subtypes Without Auditory Dysfunction. Human Gene Therapy 29(4), 492-506, Blanc et al., 2020). In particular, initial proof-of-concept of successful in vivo delivery of marker and therapeutic genes into the inner ear has been provided with AAV vector technology, and a first in-human inner ear gene therapy clinical trial using an adenoviral vector expressing ATOH1 has been launched. As the inner ear is an enclosed organ system that is filled with endolymph and perilymph, thus enabling local restriction of the vector particles upon their injection, with no spread to other tissues expected, and a distribution of the vector within this fluid upon administration, it appears a well-suited target for gene therapy. Furthermore, the number of cells to be treated is comparatively low, so that minimal vector quantities are expected to achieve a significant effect. Nevertheless, AAV and adenoviral vector systems face the limitation of a restricted coding capacity (below 5 kb), which is too small for several genes identified to be mutated in SNHL (Dong et al., 1996, Quantitative Analysis of the Packaging Capacity of Recombinant Adeno-Associated Virus. Human Gene Therapy 7, 2101-2112).

As an alternative, scientists have turned to lentiviral vectors, which have already been successfully applied in the clinics for other, non-inner-ear-related disease entities (i.e., immunodeficiencies) (Milone and O'Doherty, 2018, Clinical use of lentiviral vectors. Leukemia 32, 1529-1542). Lentiviral vectors are capable of mediating efficient gene deliveries to a variety of cell types in vitro and in vivo, and profit from increased cargo capacities of 10 kb or higher (Kumar et al., 2001, Systematic Determination of the Packaging Limit of Lentiviral Vectors. Human Gene Threapy 12, 1893-1905). Lentiviruses furthermore came into focus as potential gene vectors because of their ability to transduce non-dividing cells (Schambach et al., 2013, Biosafety Features of Lentiviral Vectors, Human Gene Therapy).

Hashimoto et al. demonstrated that lentiviral vectors may be used to treat visual symptoms of Usher syndrome in the shaker1 mouse model carrying a null mutation in the MYO7A gene. Usher syndrome is a rare genetic disorder that causes a combination of hearing loss and visual impairments in humans and may arise due to mutations in any one of at least 11 genes, amongst them MYO7A. MYO7A-deficient mice are deaf and exhibit defective melanosome localization and motility in the retinal pigment epithelium (RPE) as well as an excess of opsin in cilia of their photoreceptor cells. The authors of this study used HIV-1-derived vesicular stomatitis virus (VSV)-G-pseudotyped, 3^{rd} generation lentiviral vectors to successfully deliver cDNA of full length human MYO7A to the RPE and photoreceptor cells. At that time, the authors claimed that the 6962 bp MYO7A cDNA attached to an at least 600 bp promoter sequence constituted the largest transgene ever expressed by a viral vector in the retina. This study served as a proof-of-principle that lentiviral vectors are suitable for delivering large genes to particular cells of interest. However, the study did not address the question of whether these lentiviral vectors could also be successfully delivered to cells of the inner ear (Hashimoto et al., 2007, Lentiviral gene replacement therapy of retinas in a mouse model for Usher syndrome type 1B. Gene Therapy).

Indeed, due to the inaccessible location and the blood-labyrinth-barrier which separates the vasculature from fluids of the inner ear, successful delivery of lentiviral vectors to sensory cells of the inner ear has so far represented an insurmountable challenge.

In 1999, Han et al. attempted to deliver a second generation HIV-based, GFP-expressing and VSV-G-pseudotyped lentiviral vector via intracochlear perfusion into cells of the inner ear of guinea pigs. Their analysis of cochlear sections from these lentiviral vector-GFP-infused animals revealed fluorescence in cells bordering the periphery of the perilymphatic space into which the vector was infused as well as the spiral ligament, with fluorescence extending from the base of the cochlea to its apex with decreasing intensity. Importantly, however, the organ of Corti, which contains the sensory hair cells, and the spiral limbus were entirely free of GFP. Only upon in vitro infection of cochlear explants with lentiviral vector-GFP, did the authors observe the transduction of spiral ganglion neuros, glial cells as well as supporting cells surrounding sensory hair cells. Nevertheless, transduction of the sensory inner ear cells themselves was not detected. (Han et al., 1999, Transgene Expression in the Guinea Pig Cochlea Mediated by a Lentivirus-Derived Gene Transfer Vector. Human Gene Therapy).

Similarly, Pietola et al. injected second generation, self-inactivating, VSV-G-pseudotyped and GFP-expressing lentiviral vectors through the round window membrane (RMW) into the inner ear of mice and observed fluorescence in epithelial cells surrounding the scala vestibuli and scala tympani. Again, no GFP expression was observed in the organ of Corti and the tectorial membrane (Pietola et al., 2009, HOX-GFP and WOX-GFP lentivirus vectors for inner ear gene transfer. Acta Oto-Laryngologica).

Yan Wei et al. tested the feasibility of introducing EGFP-expressing lentiviral vectors via cochleostomy through the post-auricular route in rats. Using this delivery strategy, the authors for the first time observed EGFP fluorescence in marginal cells of the stria vascularis, the organ of Corti, the spiral nerve and spiral ganglion cells. However, successful transfection of the sensory hair cells located within the organ of Corti was not reported. In fact, injection via cochleostomy into the scala media resulted in a loss of outer hair cells at the sites of injection, which significantly impaired auditory function of the tested rats (Yan Wei et al., 2013, Effect of lentiviruses carrying enhanced green fluorescent protein injected into the scala media through a cochleostomy in rats).

Pan et al. injected a third-generation self-inactivating lentiviral vector pseudotyped with VSV-G carrying an ATOH1-eGFP transgene as cargo through the cochlear round window of rats. Based on fluorescence microscopy images showing an eGFP signal in Myosin 7A-positive inner ear cells 30 days after treatment, the authors concluded that the vector successfully infected both hair and supporting cells in the cochlea of the treated rats. In addition, gene transfer by means of the disclosed lentiviral vector was suggested to have led to a partial differentiation of supporting cells into hair cells (Pan et al., 2013, Lentivirus carrying the Atoh1 gene infects normal rat cochlea. Neural Regen Res. 8(17), 1551-1559). However, the authors did not provide any information or data regarding gene transfer efficiency. Moreover, the data presented in this study is only from selected sections of inner ear samples and no whole mounts of complete inner ears were shown. Therefore, the efficiency of the reported transduction cannot be properly assessed. Also, the authors did not provide any specification regarding the buffering system that was used for their lentiviral vector supernatants.

Collectively, the transduction rates for inner ear cell types determined in the studies described above were not sufficient to recommend the lentiviral vector technology for gene therapy against SNHL. While the use of AAV vector technology in inner ear gene therapy has delivered promising results, the lack of corresponding data for lentiviral vectors therefore has so far prevented their testing in clinical trials. Therefore, the prior art documents do not teach a lentiviral vector system that is capable of reliably transducing cells of the sensory epithelium of the cochlea, in particular both inner and outer hair cells, and that may thus come into use in human gene therapies for treating symptoms of SNHL. This problem is solved by the present invention, in particular by the subject matter of the claims.

The present invention provides a composition for use in treating sensorineural hearing loss in a subject, wherein the composition comprises a 3^{rd} generation lentiviral vector pseudotyped with a viral envelope glycoprotein capable of binding to a receptor expressed in a cell of the inner ear, wherein said composition has a titer of at least 10⁷ TU/mL and is administered to the inner ear of the subject.

The inner ear refers to the innermost part of the vertebrate ear and is responsible for sound detection and balance. The mammalian inner ear consists of the bony labyrinth comprising the cochlea, which is required for hearing, and the vestibular system that is dedicated to balance (cf. FIG. 9). The cochlea is a spiral or snail shaped structure containing three fluid filled compartments or scalae. The scala vestibuli (vestibular duct) is filled with Na⁺-rich and K⁺-low perilymph and terminates at the oval window. The scala tympani (tympanic duct) is also filled with perilymph and abuts the round window. Located between the scala vestibule and the scala tympani is the scala media (cochlear duct). It contains endolymph, which is low in Na⁺ and high in K⁺. The stria vascularis, which forms the outer wall of the scala media, pumps Na⁺ and K⁺ against their concentration gradients to create an electrical potential between endo- and perilymph known as endocochlear potential. The scala media furthermore contains the organ of Corti that sits on top of the basilar membrane, which separates the scala media from the scala tympani. The organ of Corti consists of mechanosensory epithelial cells known as inner and outer hair cells as well as the rods of Corti and a variety of supporting cells (Morgan et al., 2020, Gene therapy as a possible option to treat hereditary hearing loss. Medizinische Genetik).

When sound waves reach the outer ear, air pressure pushes against the eardrum (tympanic membrane) and induces mechanical movement of the three ossicles malleus, incus and stapes, which in turn transmit vibrations to the oval window. The pressure applied to the oval membrane triggers movement of the perilymph and endolymph within the cochlea. Motion of these inner ear fluids results in the bending of the basilar membrane and causes a plurality of stereocilia on top of the hair cells to deflect. In consequence, mechanically gated K⁺ channels open to allow small positive ions to enter, thereby causing depolarization of the hair cells. The hair cells subsequently release neurotransmitters that bind to receptors of spiral ganglion neurons (SGN). The SGN in turn fire action potentials to the brain via the cochlear nerve (Morgan et al., 2020, Gene therapy as a possible option to treat hereditary hearing loss. Medizinische Genetik).

Sensorineural hearing loss (SNHL) is a type of hearing loss that is associated with defects in the inner ear, in particular the cochlea or the vestibulocochlear nerve. SNHL can be acquired or may originate from genetic causes. Recent estimates suggest that approximately 80% of all SNHL cases arise due to genetic factors, wherein merely 20% are due to environmental causes (Blanc et al., 2020). More than 500 syndromic and non-syndromic genetic loci have been described to be associated with predispositions to hearing loss (ACMG Working Group on Update of Genetics Evaluation Guidelines for the Etiologic Diagnosis of Congenital Hearing Loss; for the Professional Practice and Guidelines Committee, 2014, American College of Medical Genetics and Genomics guideline for the clinical evaluation and etiologic diagnosis of hearing loss. Genetics in Medicine 16, 347-355). The most common form of congenital deafness in developed countries is Connexin 26 deafness (also known as GJB2-related deafness) that arises from mutations in the gene encoding Gap junction beta-2 protein (GJB2) (Cabanillas et al., 2018, Comprehensive genomic diagnosis of non-syndromic and syndromic hereditary loss in Spanish patients. BMC Medical Genomics 11, 58). Other syndromic forms of hearing impairment include Stickler syndrome, Waardenburg syndrome, Pendred syndrome or the aforementioned Usher syndrome. Congenital forms of hearing loss may also arise due to infections with rubella virus, cytomegalovirus or Toxoplasma gondii. Furthermore, the process of ageing can significantly contribute to SNHL, in particular in industrialized societies. Age-related hearing loss or Presbycusis results from the degeneration of the cochlea or associated structures of the inner ear or auditory nerves and arises due to a combination of genetic and environmental factors. The main environmental stimulus for inducing hearing loss is continuous exposure to loud noise, which gradually damages the sensory and neuronal apparatus of hearing in the inner ear. Other environmental factors that may impair hearing include the use of ototoxic drugs, including cancer chemotherapy and aminoglycoside antibiotics.

The composition of the invention may be used to effectively treat SNHL that has arisen from genetic causes. The hearing loss to be treated with the composition of the invention may for instance be caused by a mutation in a gene selected from the group comprising MYO7A, GJB2, OTOF, ATOH1, SLC26A4, TMC1, TMPRSS3, GIPC3, USH1C, RDX, CLDN14, WHRN, ESPN, ILDR1, SERPINB6, KCNQ4, POU4F3, SIX1, CCDC50, MIR96, POU3F4, EYA1, KCNQ1, GJB6, STRC, TECTA, GJB3, PRPS1, CDH23, GRXCR2, CABP2, GRXCR1, SYNE4, COL11A2, LRTOMT, BSND, PJVK, CIB2, CEACAM16, TMIE, NARS2, LHFPL5, MARVELD2, SLC22A4, KARS, SLC26A5, TPRN, ELMOD3, GPSM2, COCH, ESRRB, ADCY1, EPS8L2, CD164, OTOA, S1PR2, LOXHD1, TSPEAR, EPS8, CDC14A, MSRB3, DCDC2, PNPT1, TMEM132E, FAM65B, MYO3A, CLIC5, HGF, USH2A, TBC1D24, MET, PCDH15, PTPRQ, OTOGL, MYO6, TNC, TRIOBP, BDP1, MYO15A, DIAPH1, MYH14, DFNA5, WFS1, EYA4, COL11A2, MYH9, ACTG1, SLC17A8, GRHL2, DSPP, P2RX2, MYO1A, GFI1, TYMP and GFI1B. Accordingly, the composition of the invention may be suitable to effectively improve or even eliminate at least a symptom of a subject suffering from a genetically-induced form of hearing loss, e.g., it may restore hearing capabilities to a level similar to that of a healthy subject not suffering from genetically-induced hearing loss. In some embodiments, the composition may not be able to improve or eliminate a symptom associated with SNHL, but it may at least significantly slow down or even terminate disease progression, i.e. the symptom does not worsen over time.

The composition of the invention may also be used to treat or to prevent hearing loss in patients with haploinsufficiency or with compound heterozygous mutations.

The composition of the invention may however also effectively improve or completely eliminate at least a symptom of a subject suffering from a non-genetic form of hearing loss, e.g. by stimulating the regeneration of hair cells or auditory neurons damaged due to exposure to noise or ototoxic drugs. Alternatively, it may at least significantly slow down or even terminate disease progression. The composition may also be for use in preventing sensorineural hearing loss in a subject, e.g., by protecting cells of the inner ear from hazardous agents, such as ototoxic drugs (e.g., diuretics, non-steroidal anti-inflammatory drugs, antibiotics, chemotherapeutic agents, or quinine) or noise. Preventing includes reducing the severity of potential future sensorineural hearing loss or developing sensorineural hearing loss. The composition may also be for use in increasing survival of inner ear cells that have been damaged by such a hazardous agent. Accordingly, the composition of the invention may be for use in protecting from non-genetically-induced hearing loss, e.g., by delivery of a gene or multiple genes selected from the group comprising BDNF, GDNF, NT-3, IGF1, IGF2, CNTF, ARTN, NRTN, ATP7B, MRP1, MRP2 and MDR1. In particular, BDNF, GDNF, NT-3, IGF1, IGF2, CNTF, ARTN, and/or NRTN may stimulate regeneration or increase survival. ATP7B, MRP1, MRP2 and MDR1 may prevent damage of inner ear cells upon therapy with ototoxic drugs.

The invention likewise provides a method for treating or preventing sensorineural hearing loss in a subject in need thereof, comprising the step of administering an effective amount of the composition according to the invention to said subject.

The composition of the invention comprises a 3^{rd} generation lentiviral vector. The term “lentivirus” refers to a genus of retroviruses comprising e.g. human immunodeficiency virus (HIV), simian immunodeficiency virus (SIV), feline immunodeficiency virus (FIV) or equine infectious anemia virus (EIAV). Like that of other retroviruses, the genome of lentiviruses consists of a single-stranded (ss) positive sense RNA. During replication, the lentiviral ssRNA genome is converted into double-stranded (ds) DNA by a process known as reverse transcription. The reverse transcribed lentiviral dsDNA is subsequently integrated into the host cell's genome, which in turn replicates and transcribes the integrated lentiviral genes along with its own genes to produce new viral particles.

The lentiviral vector according to the invention may thus be derived from a lentivirus selected from the group comprising HIV-1, HIV-2, SIV, FIV or EIAV. In a preferred embodiment, the lentiviral vector is derived from HIV-1.

The lentiviral, e.g., HIV-1, genome comprises three major structural genes: gag, pol and env. gag encodes the viral matrix (MA), capsid (CA) and nucleocapsid (NC), which collectively facilitate the assembly and release of the virus particles. The pol gene encodes the viral enzymes protease (PR), reverse transcriptase (RT) and integrase (IN), which govern viral replication. The HIV-1 env encodes the viral surface glycoprotein gp160, which is subsequently cleaved to form the surface protein gp120 and the transmembrane protein gp41 during viral maturation. In addition to the structural genes gag, pol and env, the HIV-1 genome furthermore comprises the two regulatory genes tat and rev as well as the four accessory genes vif, vpr, vpu and nef. Whereas tat encodes a transactivator required for viral transcription, rev encodes a protein that controls both splicing and export of viral transcripts. The four accessory genes are considered non-essential for viral replication, but are believed to increase its efficiency (German Advisory Committee Blood (Arbeitskreis Blut), Subgroup ‘Assessment of Pathogens Transmissible by Blood’, 2016, Human Immunodeficiency Virus (HIV). Transfus Med Hemother. 43(3), 203-222).

In 1996, Naldini et al. reported the use of an HIV-based vector for in vivo gene delivery and stable transduction of non-dividing cells (Naldini et al., 1996, In vivo gene delivery and stable transduction of nondividing cells by a lentiviral vector. Science 272(5259). 263-267). Use of HIV-1-derived lentiviral vectors for in vivo gene transfer was however associated with considerable health risks. To meet these biosafety concerns, the first generation of lentiviral vector systems split the viral genome into three separate plasmids to avoid the formation of replication-competent viruses. The first plasmid encoded the actual vector that was to be integrated into the host cell's genome. It comprised the transgene of interest functionally linked to a suitable promoter sequence as well as cis-elements necessary for polyadenylation, integration, initiation of reverse transcription and packaging (e.g., the long-terminal repeats, the packaging signal, the primer binding site, the polypurine tract or the Rev-responsive element). The second plasmid, known as packaging plasmid, comprised the genes encoding the viral proteins that contribute to packaging, reverse transcription and integration of the viral genome, i.e., gag, pol, the regulatory genes tat and rev as well as the four accessory genes vif, vpu, vpr and nef. The third plasmid (Env plasmid) expressed the viral glycoprotein for host cell receptor binding. Due to the physical separation of the packaging genes from the rest of the viral genome, this split genome design prevented viral replication after infection of the host cell.

To further improve the safety of lentiviral vectors, a second generation system was established by removing all accessory genes from the packaging plasmid, as they were found to constitute crucial virulence factors.

With the third generation of lentiviral vector systems, so-called self-inactivating (SIN) vectors were introduced. When lentiviral vectors are integrated into a host cell genome, the transgene cassette of the provirus comprising the gene of interest is flanked by two long terminal repeats (LTRs). The presence of these LTRs may promote the emergence of potentially harmful replication-competent recombinants. In addition, viral promoter/enhancer regions located in the LTRs could induce the expression of adjacent host genes, with potentially tumorigenic consequences. Moreover, the promoter/enhancer regions in these LTRs can transcriptionally interfere with the promoter driving the transgene as well as the neighboring genes. Therefore, promoter/enhancer sequences of the viral 3′ LTR were removed by deleting a particular region inside the LTR (U3) from the DNA that was used to produce the viral RNA. Deletion of the U3 region effectively abolished the transcriptional activity of the LTR. Furthermore, tat, a regulatory gene driving viral transcription, was deleted from the packaging plasmid, whereas the second regulatory gene rev was provided from another separate, fourth, plasmid (Zufferey et al., 1998, Self-Inactivating Lentivirus Vector for Safe and Efficient in Vivo Gene Delivery. J Virol 72(12), 9873-9880; Schambach et al., 2013, Biosafety Features of Lentiviral Vectors. Human Gene Therapy 24, 132-142). In case the 5′ promoter driving the viral genomic mRNA is not HIV-derived and substituted by a CMV or RSV promoter, an extra tat plasmid may be omitted.

Accordingly, the lentiviral vector according to the invention is replication incompetent due to the split packaging design and self-inactivating (SIN) due to a deletion in the U3 region of the 3′ LTR. It further lacks the genes vif, vpr, vpu, nef, and, optionally, tat.

Therefore, the lentiviral vector according to the invention comprises the following features
a) a 5′ LTR comprising a constitutively active heterologous promoter at the U3 position, a repeat region (R) and a U5 region,
b) a 5′ UTR comprising a primer binding site (PBS), a splice donor site (SD), a packaging signal (ψ), a Rev-responsive element, and, optionally, a splice acceptor (SA) site,
c) an internal enhancer/promoter region operably linked to a cargo sequence,
d) RNA processing elements optionally comprising a Woodchuck hepatitis virus posttranscriptional regulatory element (PRE), and
e) a 3′ LTR with a deleted (SIN) U3 region, a repeat region (R) and a U5 region.

The splice acceptor site is optional. The splice donor site is important, but vectors lacking it are functional. Presence of RNA processing elements such as a Woodchuck hepatitis virus posttranscriptional regulatory element (PRE) contribute to efficiency, and vectors without them are typically not generated in high titers. However, they can be concentrated.

Suitable 3^{rd} generation lentiviral vectors are e.g., known in the art and/or can be prepared by the skilled person. An exemplary lentiviral vector that can be used in the invention comprises a nucleic acid sequence of SEQ ID NO: 1. The lentiviral vector may also comprise a nucleic acid sequence having at least 80%, at least 85%, at least 90%, at least 95%, at least 96%, at least 97%, at least 98%, or at least 99% sequence identity to SEQ ID NO: 1. It may also consist of SEQ ID NO: 1. Alternatively, the lentiviral vector according to the invention may comprise a different promoter region than SEQ ID NO:1, e.g., another suitable promotor as taught herein.

Optionally, the viral enhancer/promoter regions located at the U3 position of the 5′ LTR in the lentiviral vector may be replaced by a heterologous promoter to enhance expression of genomic RNA and to compensate for the loss of the lentiviral Tat protein. Therefore, the heterologous promoter preferably is a strong promoter, i.e., it drives constitutive expression of genes under its control. It may be selected from the group comprising a Rous sarcoma virus (RSV) promoter, a human cytomegalovirus (CMV) promoter, an HIV promoter (in case a lentiviral vector is used that is not derived from HIV-1 or HIV-2) or a eukaryotic promoter such as e.g., EF1a, PGK1, UBC, or human beta actin. It may also be an inducible promoter that only drives gene expression in the presence or absence of a certain stimulus, in particular, when expression of the vector would be toxic for the packaging cell. It may, e.g., be a Tet-regulated promoter, i.e., it may comprise a tetracycline response element (TRE) that can be bound by a tetracycline transactivator (tTA) protein in the presence of tetracycline or an analogue thereof, e.g. doxycycline. Preferably, the heterologous promoter is a constitutively active promoter derived from RSV or CMV.

The 5′ UTR packaging signal (ψ) interacts with the nucleocapsid (NC) of the assembling Gag proteins to mediate packaging. Optimal packaging furthermore requires the trans-activation response element (TAR), U5, the primer binding site (PBS) and the group-specific antigen (Gag). The Rev protein additionally enhances the encapsidation of RNA containing the Rev-responsive element (RRE). The RRE also allows for higher expression of the viral genome and may suppress aberrant splice events (Schambach et al., 2013). The 5′ UTR may, optionally, further comprise multiple splice sites. In particular, the lentiviral vector may comprise a 5′UTR splice donor (SD) site and, optionally, a splice acceptor site (SA). In an optional embodiment, the 5′ UTR may further comprise a polypurine tract (PPT) region, preferably downstream of the RRE. The PPT region is thought to increase the efficiency of reverse transcription and may even mediate nuclear entry of the lentiviral pre-integration complex, which forms after uncoating of the viral particle upon entry into the host cell (Schambach et al., 2013).

The internal promoter/enhancer region is preferably a strong promoter, i.e., it drives constitutive expression of the cargo sequence under its control. It may be a promoter selected from the group comprising a viral promoter, a cellular promoter, a cell-specific promoter, an inducible promoter or a synthetic promoter.

It may, e.g., be a viral promoter selected from the group comprising a promoter derived from CMV, spleen focus-forming virus (SFFV), myeloproliferative sarcoma virus (MPSV), murine embryonal stem cell virus (MESV), murine leukemia virus (MLV) and simian virus 40 (SV40). Preferably, the internal promoter is a promoter derived from CMV or SFFV, most preferably from CMV. In another preferred embodiment, the promoter may also be a CAG promoter, i.e. a composite construct consisting of the CMV enhancer fused to the chicken beta-actin promoter and the rabbit beta-Globin splice acceptor site. The internal promoter/enhancer region may alternatively be a cellular promoter selected from the group comprising EF1a, PGK1 and UBC. It may also be a promoter that enables expression of the cargo sequence in a cell-specific manner. It may, e.g., be a hair cell-specific promoter selected from the group comprising POU4F3, POU3F4, ATOH1, Prestin, Pendrin and MYO7A or an SGN-specific promoter selected from the group comprising e.g. MAP1B, SYN, and NSE.

The internal promoter/enhancer may also be an inducible promoter, especially, if expression of the cargo sequence may lead to cytotoxicity. The promoter may thus be selected from the group comprising a TET-inducible promoter, a Cumate-inducible promoter, an electrosensitive promoter and an optogenetic switch. Electrosensitive promoters or optogenetic switches may be especially useful when expression of the cargo sequence is to be controlled by a cochlea implant.

The internal promoter/enhancer region may also be a hybrid or a synthetic promoter selected from the group comprising CAG and MND. A hybrid promoter is composed of several regulatory elements from different promoters/enhancers, which are joined to build a new promoter combining the desired features. In contrast, a synthetic promoter essentially consists of an array of transcription factor binding sites fused to a minimal promoter. A preferred hybrid promoter used for driving cargo sequence expression is CAG.

Finally, the internal enhancer/promoter region may comprise a chromatin opening element, i.e., a DNA sequence consisting of methylation-free CpG islands that either encompasses a housekeeping gene promoter or is operably linked to a heterologous promoter to confer reproducible, stable transgene expression.

The 3′ UTR of the lentiviral vector according to the invention may comprise an RNA processing element, e.g., a Woodchuck hepatitis virus posttranscriptional regulatory element (PRE). The PRE may enhance cargo sequence expression by up to 10-fold and contributes to high virus titers. It also improves mRNA stability as well as translation efficiency and reduces read-through transcription into adjacent cellular sequences (Schambach et al., 2013).

To further prevent read-through transcription, the 3′ end of the lentiviral vector may comprise upstream sequence elements (USE) that can be inserted into the 3′ U3 region to improve 3′ end processing and transcriptional termination. Preferably, the USE is a recombinant direct repeat of the USE derived from SV40 (2×SV USE). The 3′ UTR may, optionally, further comprise target sites for differentiation-specific cellular miRNAs to enable post-transcriptional targeting of the vector mRNA.

Upon random or semi-random integration of a lentiviral vector into or next to a host cell's gene, the vector may act as a mutagen that dysregulates the gene's expression. In an optional embodiment, the space created by deletion of the U3 region in the 3′LTR of the lentiviral vector may therefore comprise an insulator sequence. Insulator sequences constitute barriers between regulator gene regions and thus may prevent vector-driven dysregulation of adjacent proto-oncogenes, i.e., cellular genes whose erroneous expression may provoke tumorigenesis (Schambach et al., 2013).

To further increase biosafety of the composition according to the invention, the packaging plasmid encoding for Gag-Pol may be designed to comprise an inhibitory mutation in the pol region encoding for the integrase protein to render the lentiviral vector integration-deficient. Possible mutations may alter a triad of amino acid residues at the catalytic core of the integrase enzyme, e.g., D64, D116 and E152. Alternatively, amino acid changes may be introduced into the integrase DNA attachment site e.g. by mutating nucleotides within a 12 base-pair (bp) region of the vector 5′ LTR U3 region or an 11 bp region of the 3′LTR U5 region (Shaw and Cornetta, 2014, Design and Potential of Non-Integrating Lentiviral Vectors. Biomedicines 2(1), 14-35). Therefore, in an optional embodiment, the lentiviral vector according to the invention is a non-integrating lentiviral vector.

The lentiviral vector of the composition for use according to the invention furthermore comprises a cargo sequence, wherein expression of said cargo sequence in said cell of the inner ear is capable of reducing, eliminating or preventing at least a symptom of sensorineural hearing loss in the subject. The cargo sequence is operably linked to the internal promoter/enhancer region. It comprises a nucleic acid selected from the group comprising a protein-coding gene, a miRNA, an shRNA or a lncRNA.

Preferably, the cargo sequence is a protein-coding gene, wherein a reduced or erroneous expression of said gene is associated with the development of the sensorineural hearing loss in the subject. Accordingly, the cargo sequence may be an intact copy of any gene known to drive SNHL when being mutated. It may, for instance, be a gene such as MYO7A, GJB2, OTOF, ATOH1, SLC26A4, TMC1, TMPRSS3, GIPC3, USH1C, RDX, CLDN14, WHRN, ESPN, ILDR1, SERPINB6, KCNQ4, POU4F3, SIX1, CCDC50, MIR96, POU3F4, EYA1, KCNQ1, GJB6, STRC, TECTA, GJB3, PRPS1, CDH23, GRXCR2, CABP2, GRXCR1, SYNE4, COL11A2, LRTOMT, BSND, PJVK, CIB2, CEACAM16, TMIE, NARS2, LHFPL5, MARVELD2, SLC22A4, KARS, SLC26A5, TPRN, ELMOD3, GPSM2, COCH, ESRRB, ADCY1, EPS8L2, CD164, OTOA, S1PR2, LOXHD1, TSPEAR, EPS8, CDC14A, MSRB3, DCDC2, PNPT1, TMEM132E, FAM65B, MYO3A, CLIC5, HGF, USH2A, TBC1D24, MET, PCDH15, PTPRQ, OTOGL, MYO6, TNC, TRIOBP, BDP1, MYO15A, DIAPH1, MYH14, DFNA5, WFS1, EYA4, COL11A2, MYH9, ACTG1, SLC17A8, GRHL2, DSPP, P2RX2, MYO1A, GFI1, TYMP, or GFI1B.

The gene may, e.g., be a transcription factor or a combination of several different transcription factors that induce(s) transdifferentiation of a given cell into a cell required for hearing. For example, expression of one or more transcription factors in the inner ear may induce the differentiation of glial cells within the organ of Corti into auditory neurons. Alternatively, expression of one or more transcription factor(s) such as, e.g., Atoh1, Gfi1, Pou4f3 or Pou3f4 may induce differentiation of non-sensory auditory epithelial cells into new functional hair cells. Similarly, expression of the transcription factors Sox2, NeuroG1 or NeuroD1 may drive the differentiation of cells into SGN. For this purpose, the composition of the invention may furthermore comprise growth factors, e.g. a neurotrophic factor such as BDNF, GDNF, NT3 or IGF. Alternatively, the cargo sequence may encode said growth factors.

Under certain circumstances, it may be beneficial to inactivate the translated protein encoded by the cargo sequence after a given time, e.g., due to arising cytotoxicity. Therefore, the coding sequence may be designed to further encode an instable degradation or destabilization domain. The cargo sequence may for instance encode a rapidly degradable E. coli-derived dihydrofolate reductase (ecDHFR)-domain fused to the protein of interest. The ecDHFR-domain can be stabilized in a reversible and dose-dependent manner by the small antibiotic ligand trimethoprim (TMP). Upon withholding TMP from the transduced cell, the instability of the ecDHFR-domain is conferred to the fused protein of interest, causing rapid degradation of the entire fusion protein (Iwamoto et al., 2010, A general chemical method to regulate protein stability in the mammalian central nervous system. Chem Biol. 17(9), 981-988). A person skilled in the art may select another destabilization domain, e.g. that has been engineered in the past (derived, e.g., from DHFR, FRB or FKBP12) and that may be similarly used to control the stability of the protein encoded by the cargo sequence (Lévy et al., 1999, Analysis of a conditional degradation signal in yeast and mammalian cells. Eur J Biochem. 259(1-2), 244-252; Stankunas et al., 2003, Conditional protein alleles using knockin mice and a chemical inducer of dimerization. Mol Cell. 12(6), 1615-1624).

The cargo sequence preferably is a complementary DNA (cDNA) that has been generated via reverse transcription from messenger RNA (mRNA) transcribed from the gene of interest and that lacks introns normally present in the gene of interest. Alternatively, it may be the complete gene of interest comprising introns and, optionally, regulatory regions provided that the length of the cargo sequence does not exceed the packaging limit of the lentiviral vector. The cargo sequence may alternatively be synthetically engineered, it may, e.g., be codon-optimized to ensure efficient expression of the gene of interest in the subject, e.g., codon-optimized for expression in human cells. The cargo sequence may, optionally, further comprise an internal ribosomal entry site (IRES), i.e. an RNA element that mediates mRNA translation in the absence of a 5′ cap and allows for co-expression of multiple genes under the control of a single promoter.

In an alternative embodiment, the cargo sequence may be transcribed into a regulatory RNA selected from the group comprising a microRNA, a short hairpin RNA or a long non-coding RNA. Preferably, the cargo sequence is transcribed into a microRNA (miRNA), i.e., a regulatory RNA molecule that is originally transcribed as a hairpin stem loop before being processed into a short single-stranded 20-25 nt RNA molecule capable of post-transcriptionally silencing gene expression. Endogenously transcribed miRNAs are estimated to regulate more than 60% of all protein coding genes and to modulate virtually every cellular pathway (Catalanotto et al., 2016, MicroRNA in Control of Gene Expression: An Overview of Nuclear Functions. International journal of molecular sciences, 17(10), 1712). Therefore, the cargo regulatory RNA, e.g., miRNA, according to the invention may be an RNA whose reduced or erroneous expression is associated with the development of SNHL in a subject. Alternatively, the cargo regulatory RNA, e.g., miRNA, according to the invention may also be an RNA that targets a gene whose elevated or erroneous expression is associated with the development of SNHL in a subject.

In yet another embodiment, the cargo sequence of the invention may be transcribed into a short hairpin RNA (shRNA). shRNAs are synthetic RNA molecules that, similar to miRNAs, can silence gene expression via RNA interference. The cargo shRNA according to the invention may target a gene whose elevated or erroneous expression is associated with the development of the sensorineural hearing loss in the subject.

Alternatively, the cargo sequence may also be transcribed into a long non-coding RNA (lncRNA). LncRNAs are non-coding transcripts larger than 200 nt that may fulfill diverse functions, e.g., in regulation of gene transcription, splicing, translation or epigenetic regulation. The cargo lncRNA according to the invention may be a lncRNA whose reduced or erroneous expression is associated with the development of the sensorineural hearing loss in the subject, or wherein said lncRNA may regulate expression of a gene whose elevated or erroneous expression is associated with the development of the sensorineural hearing loss in the subject.

In yet another embodiment, the cargo sequence may encode a Cas, e.g., Cas9 protein, and further comprise a single guide RNA (sgRNA) for efficient CRISPR/Cas delivery to inner ear cells. The clustered regularly interspaced palindromic repeats (CRISPR)/CRISPR associated protein (Cas), e.g., Cas9 system may be used for targeted genome-editing. The expressed sgRNA finds and binds to a specific sequence in the host cell's DNA and the Cas enzyme subsequently introduces double or single stranded cuts into specific adjacent DNA regions. Repair of these cuts may result in target gene knock-out due to introduction of insertions or deletions. Alternatively, the system may be used to introduce a stretch of DNA into the cutting site, e.g. an intact copy of a mutated gene.

In one embodiment, expression of the cargo sequence in a cell of the inner ear may also treat or prevent hearing loss arising from a non-genetic cause. Cargo sequence expression in a cell of the inner ear may, for instance, compensate for the loss of hair cells due to infection or exposure to loud noise, ototoxic drugs or mechanical trauma. The cargo sequence may, e.g., be a gene or multiple genes selected from the group comprising BDNF, GDNF, NT-3, IGF1, IGF2, CNTF, ARTN, and NRTN. It may also be a regulatory RNA (e.g., a miRNA) that cell-specifically silences the expression of a gene that normally prevents regeneration of sensory hair cells in the organ of Corti. It may, e.g., be a miRNA that targets the retinoblastoma gene (Rb1), which was reported to maintain hair cells and supporting cells in a post-mitotic state. Previous reports have demonstrated that targeted deletion of Rb1 may trigger proliferation of functional hair cells in vivo (Sage et al., 2005, Proliferation of functional hair cells in vivo in the absence of the retinoblastoma protein. Science 307(5712), 1056-1058.). The cargo sequence may also encode, e.g., a transcription factor that induces transdifferentiation of cells into sensory hair cells or SGNs, a growth factor, or a molecular drug pump/transporter that actively exports and thereby removes a hazardous agent, e.g., an ototoxic drug from a cell of the inner ear, e.g., a gene or multiple genes selected from the group comprising ATP7B, MRP1, MRP2 and MDR1.

Preferably, expression of the cargo sequence according to the invention in a cell of the inner ear is capable of reducing, eliminating or preventing at least a symptom of sensorineural hearing loss in the subject. In the context of the invention, “reducing a symptom” means that the severity of a symptom may be alleviated, e.g. upon administration of the composition, the condition of a subject with severe hearing impairments may be ameliorated to an extent to which his/her hearing capabilities have been significantly improved compared to the hearing capabilities prior to administration. In another embodiment, a symptom of sensorineural hearing loss may even be completely eliminated in a subject upon expression of the cargo sequence, i.e., the subject suffering from SNHL may have his/her hearing capabilities fully restored upon administration of the composition according to the invention. In yet another embodiment, the expression of the cargo sequence may prevent a symptom of SNHL in a subject, e.g., completely prevent or reduce the incidence or severity of said symptom. For instance, the composition according to the invention may be administered to a fetus that has been diagnosed with a genetic deficiency associated with a later on-set development of SNHL. Expression of the cargo sequence may compensate for the genetic defects and thus prevent the emergence of SNHL in later life. Preventing a symptom of SNHL may also refer to protecting a subject from the hazardous effects of external stimuli. Preventing includes reducing the severity and/or incidence of potential future sensorineural hearing loss or developing sensorineural hearing loss. Expression of the cargo sequence in a sensory hair cell or auditory neuronal cell may, e.g., confer resistance to specific ototoxic drugs that are employed during chemotherapeutic treatment of a cancer patient.

The lentiviral vector according to the invention is pseudotyped with a viral envelope glycoprotein capable of binding to a receptor expressed in a cell of the inner ear.

The term pseudotyping refers to the generation of viral vectors that carry foreign viral envelope proteins on their surface. Viral surface glycoproteins modulate viral entry into the host cell by interacting with particular cellular receptors to induce membrane fusion. For instance, the native HIV envelope glycoprotein specifically binds the receptor CD4 as well as co-receptors CXCR4 or CCR5, which effectively limits the potential target cells of HIV to CD4⁺ T-cells and monocytes. To change or broaden the selection of cells that may be transduced, lentiviral vectors are therefore usually pseudotyped with heterologous envelope glycoproteins both from related and unrelated viruses. Dependent on the choice of glycoprotein used for pseudotyping, the tropism of the virus, i.e., the range of host cells that can be infected by the lentivirus, can thus be either expanded or directed to particular cell types of interest.

Preferably, in the context of the invention, the lentiviral vector is pseudotyped with a viral envelope glycoprotein capable of binding to a receptor selected from the group consisting of the LDL-receptor and LDL-R family members, the SLC1A5-receptor, the Pit1/2-receptor and the PIRYV-G-receptor.

Most preferably, said receptor is an LDL-receptor or LDL-R family member, e.g., an LDL-receptor.

The evolutionary conserved Low-Density Lipoprotein (LDL) receptor is a cell surface receptor that mediates endocytosis of cholesterol-rich LDL. It is ubiquitously expressed in all nucleated cells, in particular inside the liver where it removes the majority of LDL from the circulation. In addition, the LDL-receptor serves as an entry site for a wide variety of viruses, including Hepatitis C virus (Agnello et al., 1999, Hepatitis C virus and other flaviviridae viruses enter cells via a low density lipoprotein receptor. Proc Natl Acad Sci 96(22), 12766-12771). In 2013, Finkelshtein et al. identified the LDL-receptor as the major entry port of vesicular stomatitis virus (VSV), which explained why viruses and viral vectors pseudotyped with the VSV envelope glycoprotein G (VSV-G) displayed an extraordinarily broad tropism (Finkelshtein et al., 2013, LDL receptor and its family members serve as the cellular receptors for vesicular stomatitis virus. PNAS 110(18), 7306-7311). The LDL-receptor family member megalin was found to be expressed in various cell types of the cochlea. However, no evidence for megalin expression was found in SGNs or inner and outer hair cells or supporting cells in the organ of Corti (Hosokawa et al., 2018, Immunohistochemical localization of megalin and cubilin in the human inner ear. Brain Res. 1701, 153-160). The present inventors unexpectedly found that the LDL receptor is expressed in inner ear cells, for example, in inner and outer hair cells as well as in SGNs. Accordingly, these cells can be transduced by viruses pseudotyped with VSV-G.

The viral envelope glycoprotein capable of binding the LDL-receptor or LDL-R family members may be, e.g., MARAV-G, COCV-G, VSV-G or VSV-G ts.

Lentiviral vectors are most commonly pseudotyped with vesicular stomatitis virus envelope glycoprotein (VSV-G). As the broadly expressed LDL-receptor and other LDL-receptor family members serve as entry sites for VSV, the VSV-G envelope confers a very broad tropism to viral vectors. Upon binding of VSV-G to the LDL-receptor, the virion is taken up by the host cell via clathrin-mediated endocytosis. Besides providing high tropism, VSV-G furthermore significantly increases the stability of the lentiviral vector, which allows for efficient concentration of viral titers by centrifugation, another essential feature for effective, virus-mediated gene transfer. Accordingly, pseudotyping of lentiviral vectors with VSV-G allows for robust transduction into a wide range of different cell types. Using the composition of the invention, the inventors were able to transduce the auditory, hair-cell-like cell line HEI-OC1 as well as primary dissociated SGNs with VSV-G-pseudotyped lentiviral vectors. The inventors could further prevent progressive hearing loss in a MYO7A-deficient shaker-1 mouse model by injecting a VSV-G-pseudotyped lentiviral vector into the vestibular labyrinth of the inner ear. Subsequent analysis revealed, for the first time, successful and highly efficient transduction of cochlear cells, including inner and outer hair cells and SGNs with lentiviral vectors. Therefore, in one embodiment, the lentiviral vector of the invention is pseudotyped with VSV-G, e.g., wild-type VSV-G or a VSV-G derivative capable of binding to the LDL-receptor or LDL-R family members. Preferably, the wild type VSV-G is a glycoprotein derived from the Indiana VSV serotype and has an amino acid sequence having at least 80%, preferably at least 85%, at least 90%, at least 95%, at least 96%, at least 97%, at least 98%, at least 99% or 100% sequence identity to SEQ ID NO: 21. Optionally, it is encoded by a nucleic acid sequence having at least 80%, preferably at least 85%, at least 90%, at least 95%, at least 96%, at least 97%, at least 98%, at least 99% or 100% sequence identity to SEQ ID NO: 20. It may also be derived from the New Jersey VSV serotype and have an amino acid sequence having at least 80%, preferably at least 85%, at least 90%, at least 95%, at least 96%, at least 97%, at least 98%, at least 99% or 100% sequence identity to SEQ ID NO: 23. Optionally, it is encoded by a nucleic acid sequence having at least 80%, preferably at least 85%, at least 90%, at least 95%, at least 96%, at least 97%, at least 98%, at least 99% or 100% sequence identity to SEQ ID NO: 22. To achieve higher particle stability upon in-vivo administration and to evade potential recognition by the host's complement system, a thermostable and complement-resistant VSV-G glycoprotein (VSV-G ts) may alternatively be used, which may have an amino acid sequence having at least 80%, preferably at least 85%, at least 90%, at least 95%, at least 96%, at least 97%, at least 98%, at least 99% or 100% sequence identity to SEQ ID NO: 3. It may, optionally, be encoded by a nucleic acid sequence having at least 80%, preferably at least 85%, at least 90%, at least 95%, at least 96%, at least 97%, at least 98%, at least 99% or 100% sequence identity to SEQ ID NO: 2, and capable of binding to the LDL-R or LDL-R family members.

However, in a preferred embodiment, the lentiviral vector according to the invention is pseudotyped with a COCV-G glycoprotein, i.e., a glycoprotein derived from Cocal virus. COCV-G is capable of binding to the LDL-receptor and it may, e.g., have an amino acid sequence having at least 80%, preferably at least 85%, at least 90%, at least 95%, at least 96%, at least 97%, at least 98%, at least 99% or 100% sequence identity to SEQ ID NO: 5. Optionally, COCV-G may be encoded by a nucleic acid sequence having at least 80%, preferably at least 85%, at least 90%, at least 95%, at least 96%, at least 97%, at least 98%, at least 99% or 100% sequence identity to SEQ ID NO: 4. In 2010, Trobridge et al. addressed some of the disadvantages of VSV-G-pseudotyped lentiviral vectors, including the aforementioned cytotoxicity associated with constitutive VSV-G expression, but also the inactivation of VSV-G-pseudotyped vectors by human serum complement. The authors of this study demonstrated that lentiviral vectors pseudotyped with COCV-G glycoprotein derived from Cocal virus could be produced at titers as high as VSV-G-pseudotyped vectors, and exhibited both broad tropism and high stability. Importantly, pseudotyping with COCV-G also confers resistance to viral inactivation by human serum (Trobridge et al., 2010, Cocal-pseudotyped Lentiviral Vectors Resist Inactivation by Human Serum and Efficiently Transduce Primate Hematopoietic Repopulating Cells. Molecular Therapy 18(4), 725-733). The present inventors made the surprising discovery that lentiviral vectors pseudotyped with COCV-G transduced the murine inner-ear-derived hair-cell-like cell line HEI-OC1 with even higher efficiency than vectors pseudotyped with VSV-G (FIG. 4).

In a particularly preferred embodiment, the glycoprotein used for pseudotyping the lentiviral vector of the invention capable of binding to the LDL-receptor is MARAV-G. MARAV-G may, e.g., have an amino acid sequence having at least 80%, preferably at least 85%, at least 90%, at least 95%, at least 96%, at least 97%, at least 98%, at least 99% or 100% sequence identity to SEQ ID NO: 7. Optionally, it may be encoded by a nucleic acid sequence having at least 80%, preferably at least 85%, at least 90%, at least 95%, at least 96%, at least 97%, at least 98%, at least 99% or 100% sequence identity to SEQ ID NO: 6. MARAV-G is an envelope glycoprotein derived from Maraba virus. Maraba virus is a rhabdovirus best known for displaying broad tropism for both human and murine cancer cells. The inventors could show that the use of lentiviral vectors pseudotyped with MARAV-G allows for an unexpectedly efficient transduction of both HEI-OC1 cells as well as dissociated primary SGNs. In fact, in both cell types, MARAV-G-pseudotyped lentiviral vectors performed significantly better than their VSV-G- or COCV-G-pseudotyped counterparts (FIG. 4 and FIG. 7). In addition, lentiviral vectors according to the invention pseudotyped with the MARAV-G glycoprotein appeared to transduce more layers of cells (i.e. also deeper layers) within the stria vascularis upon their in vivo injection into normal hearing mice than VSV-G or COCV-G-pseudotyped vectors (FIG. 12).

Alternatively, the lentiviral vector according to the invention may also be pseudotyped with a viral envelope glycoprotein capable of binding the SLC1A5-receptor. SLC1A5 was originally identified as a neutral amino acid transporter that exchanges glutamine with other neutral amino acids such as serine, asparagine or threonine in a Na⁺-dependent manner (Scalise et al., 2018, The Human SLC1A5 (ASCT2) Amino Acid Transporter: From Function to Structure and Role in Cell Biology. Front Cell Dev Biol.). Expression of SLC1A5 was reported for lung, skeletal muscle, large intestine, kidney, testis, T-cells, brain and adipose tissue (Scalise et al., 2018). SLC1A5 is, however, also used as a common entry port by a group of retroviruses, including the feline endogenous virus (RD114) and baboon endogenous virus (BaEV) (Marin et al., 2003, N-Linked Glycosylation and Sequence Changes in a Critical Negative Control Region of the ASCT1 and ASCT2 Neutral Amino Acid Transporters Determine Their Retroviral Receptor Functions. Journal of Virology 77(5), 2936-2945).

Accordingly, the lentiviral vector of the invention may also be pseudotyped with a viral envelope glycoprotein derived from RD114 glycoprotein (GP) that is capable of binding the SLC1A5-receptor. Such a glycoprotein may have an amino acid sequence having at least 80%, preferably at least 85%, at least 90%, at least 95%, at least 96%, at least 97%, at least 98%, at least 99% or 100% sequence identity to SEQ ID NO: 9. Optionally, it may be encoded by a nucleic acid sequence having at least 80%, preferably at least 85%, at least 90%, at least 95%, at least 96%, at least 97%, at least 98%, at least 99% or 100% sequence identity to SEQ ID NO: 8. It thus may also be RD114 GP. A suitable RD114 GP derivative may, e.g., be RD114\TR, which has been modified to comprise a cytoplasmic tail (designated TR) of amphotropic murine leukemia virus (MLV-A) glycoprotein in its intracytoplasmic domain (Sandrin et al., 2002, Lentiviral vectors pseudotyped with a modified RD114 envelope glycoprotein show increased stability in sera and augmented transduction of primary lymphocytes and CD34+ cells derived from human and nonhuman primates. Blood 100(3), 823-32). It may also be a glycoprotein derived from BaEV GP that is capable of binding the SLC1A5-receptor. Such a glycoprotein may, e.g., have an amino acid sequence having at least 80%, preferably at least 85%, at least 90%, at least 95%, at least 96%, at least 97%, at least 98%, at least 99% or 100% sequence identity to SEQ ID NO: 11. Optionally, it may be encoded by a nucleic acid sequence having at least 80%, preferably at least 85%, at least 90%, at least 95%, at least 96%, at least 97%, at least 98%, at least 99% or 100% sequence identity to SEQ ID NO: 10. It thus may also be BaEV GP. Suitable BaEV GP derivatives may, e.g., be BaEV\TR or BaEV\Rless. The latter harbors a specific deletion of the R peptide sequence in its cytoplasmic tail (Girard-Gagnepain et al., 2014, Baboon envelope pseudotyped LVs outperform VSV-G-LVs for gene transfer into early-cytokine-stimulated and resting HSCs. Blood 124(8), 1221-1231).

The lentiviral vector of the invention may also be pseudotyped with a viral envelope glycoprotein capable of binding the Pit1/2-receptor. Pit1 and Pit2 are sodium-dependent phosphate transporters that play a vital role in phosphate transport to ensure normal cellular function. Pit1 and Pit2 serve also as receptors for the gibbon ape leukemia virus (GALV) and the amphotropic murine leukemia virus (A-MuLV), respectively (Leverett et al., 1998, Entry of amphotropic murine leukemia virus is influenced by residues in the putative second extracellular domain of its receptor, Pit2. J Virol. 72(6), 4956-61). Therefore, the viral envelope glycoprotein may be derived from GALV. GALV GP is capable of binding the Pit1/2-receptor, and it may, e.g., have an amino acid sequence having at least 80%, preferably at least 85%, at least 90%, at least 95%, at least 96%, at least 97%, at least 98%, at least 99% or 100% sequence identity to SEQ ID NO: 13. Optionally, it may be encoded by a nucleic acid sequence having at least 80%, preferably at least 85%, at least 90%, at least 95%, at least 96%, at least 97%, at least 98%, at least 99% or 100% sequence identity to SEQ ID NO: 12. The viral glycoprotein may be alternatively derived from A-MuLV/Ampho. Such an Ampho GP is capable of binding the Pit1/2-receptor, and it may, e.g., have an amino acid sequence having at least 80%, preferably at least 85%, at least 90%, at least 95%, at least 96%, at least 97%, at least 98%, at least 99% or 100% sequence identity to SEQ ID NO: 15. Optionally, it may be encoded by a nucleic acid sequence having at least 80%, preferably at least 85%, at least 90%, at least 95%, at least 96%, at least 97%, at least 98%, at least 99% or 100% sequence identity to SEQ ID NO: 14. It may also be pseudotyped with a glycoprotein capable of binding the Pit1/2-receptor and derived from 10A1 MLV having an amino acid sequence having at least 80%, preferably at least 85%, at least 90%, at least 95%, at least 96%, at least 97%, at least 98%, at least 99% or 100% sequence identity to SEQ ID NO: 17. Thus, the glycoprotein may, optionally, be encoded by a nucleic acid sequence having at least 80%, preferably at least 85%, at least 90%, at least 95%, at least 96%, at least 97%, at least 98%, at least 99% or 100% sequence identity to SEQ ID NO: 16.

The lentiviral vector of the invention may be alternatively pseudotyped with PIRYV-G having an amino acid sequence having at least 80%, preferably at least 85%, at least 90%, at least 95%, at least 96%, at least 97%, at least 98%, at least 99% or 100% sequence identity to SEQ ID NO: 19. Optionally, it may be encoded by a nucleic acid sequence having at least 80%, preferably at least 85%, at least 90%, at least 95%, at least 96%, at least 97%, at least 98%, at least 99% or 100% sequence identity to SEQ ID NO: 18. The glycoprotein is thus capable of mediating entry into a host cell that can be entered by PIRYV-G having SEQ ID NO: 19. PIRYV-G is derived from the Piry virus, which, like VSV, MARAV and COCV, belongs to the genus vesiculovirus. Similar to COCV-G, PIRYV-G was shown to be more resistant to complement-mediated inactivation by mammalian sera than VSV-G (Tijani et al., 2018, Lentivector Producer Cell Lines with Stably Expressed Vesiculovirus Envelopes. Mol Ther Methods Clin Dev. 10, 303-312). However, the cellular surface receptor facilitating the entry of Piry virus into a host cell is currently unknown.

Optionally, the lentiviral vector may be pseudotyped with more than one type of heterologous glycoprotein. It may, e.g., be pseudotyped with two different glycoproteins targeting the same receptor protein, such as VSV-G and COCV-G. Alternatively, the lentiviral vector may be pseudotyped with a mixture of glycoproteins that bind to at least two, i.e. two, three or four different receptor proteins. It may, e.g., be simultaneously pseudotyped with COCV-G binding to the LDL receptor and RD114 binding to SLC1A5.

The composition of the invention is obtainable from packaging the lentiviral vector, preferably, in a process comprising providing at least four distinct expression plasmids. The lentiviral particles may be provided from a vector plasmid encoding the lentiviral vector genome itself as described above, a packaging plasmid coding for Gag and Pol, a plasmid encoding Rev and a plasmid encoding at least one of the herein mentioned envelope glycoproteins, e.g., as disclosed by Schambach et al., 2006 (Equal potency of gammaretroviral and lentiviral SIN vectors for expression of 06-methylguanine-DNA methyltransferase in hematopoietic cells. Mol. Ther. 13(2), 391-400). The vector plasmid may e.g. comprise a nucleic acid sequence of SEQ ID NO: 24 or having at least 80%, at least 85%, at least 90%, at least 95%, at least 96%, at least 97%, at least 98%, or at least 99% sequence identity thereto. Different promotor and/or enhancer and/or resistance elements may be used if desired. The packaging plasmid may e.g. comprise a nucleic acid sequence of SEQ ID NO: 25, or having at least 80%, at least 85%, at least 90%, at least 95%, at least 96%, at least 97%, at least 98%, or at least 99% sequence identity thereto. A different promotor may also be used. The Rev-encoding plasmid may e.g. comprise a nucleic acid sequence of SEQ ID NO: 26 or having at least 80%, at least 85%, at least 90%, at least 95%, at least 96%, at least 97%, at least 98%, or at least 99% sequence identity thereto. The Env-encoding plasmid may e.g. comprise a nucleic acid sequence of SEQ ID NO: 27 or having at least 80%, at least 85%, at least 90%, at least 95%, at least 96%, at least 97%, at least 98%, or at least 99% sequence identity thereto. A different promotor may also be used. Suitable promotors or other functional elements are e.g., taught herein.

Because the split genome design of lentiviral vectors effectively prevents the replication of lentiviral vectors, they need to be generated by co-expressing the different viral constituents in a single packaging cell. The at least four distinct expression plasmids are therefore co-transfected into a packaging cell to produce the lentiviral vector at sufficiently high titer. The packaging cell may be selected from the group comprising a HEK293T cell, a HEK293 cell, a NIH3T3 cell, a HeLa cell, an HT 1080 cell, a COS-1 cell and an AGE1.CR cell. Preferably, the packaging cell is a human cell, most preferably a HEK293T or HEK293 cell. The ratio of the Gag/Pol encoding packaging construct, the Env plasmid, the Rev plasmid and the vector plasmid may be 10-20:1-10:5-10:5:10, preferably 13-16:1-7:6-8:6-8. Especially the amount of Env-encoding plasmid may vary considerably depending on the selection of the glycoprotein for lentiviral vector pseudotyping. The medium comprising the produced lentiviral particles may be harvested 25-40 h, e.g. 30-36 h post-transfection of the packaging cells. Optionally, a second harvest of newly generated lentiviral particles may be performed 40-60 h, e.g. 48-54 h post-transfection. Preferably, the medium comprising the lentiviral particles is filtered using a 0.22 μm pore size filter. Media comprising virus particles obtained from subsequent harvests may be pooled and, optionally, stored, e.g., at −80° C.

Co-expression of multiple plasmids in a single packaging cell such as a HEK293T cell according to the invention enables the production of lentiviral particles at high titers. Because the inner ear is a rather small and highly secluded organ that is difficult to access, only small volumes of the composition according to the invention can be applied to a subject (up to about 20 μL for humans and about 1 μL for mice). The efficiency of gene transfer and in turn of gene expression, however, largely depends on the total number of vector particles delivered per target cell (Zhang et al, 2001). The composition of the invention, therefore, needs to possess a virus titer high enough to deliver a sufficient amount of lentiviral particles to facilitate effective transduction of inner ear cells. The viral titer of a composition is most accurately provided as functional titer, i.e. it describes how many viral particles have actually infected a target cell. It is, therefore, also designated an infectious titer. It is commonly expressed as transduction units per mL (TU/mL) and can be assessed after transduction of a cell, e.g., by PCR-based methods or flow cytometry. The final virus titer of the present composition should be of a magnitude of at least 10⁶, preferably of at least 10⁷, most preferably of at least 10⁸ TU/mL. It may even be higher, e.g., at least 10⁹ TU/mL or even at least 10¹⁰ TU/mL.

To reach a functional titer of the above mentioned magnitude, the lentiviral vector titer of the composition according to the invention may be further concentrated after harvesting of the lentiviral particles from packaging cells, e.g., using ultracentifugation. In a preferred embodiment, the lentiviral vector titer is concentrated using ultracentrifugation, wherein the viral vector particles are centrifuged at at least 10,000×g for at least 1 h, at least 1.5 h or, preferably, for at least 2 h, at least 2.5 h, at least 3 h, at least 3.5 h or at least 4 h (Zhang et al., 2001,). The speed and duration of centrifugation depend on the respective pseudotype of the viral vector particle. VSV-G-pseudotyped lentiviral vector particles may, for instance, be centrifuged at 82,740×g for 2 h. In contrast, vector particles pseudotyped with RD114, GALV or BaEV may be centrifuged at about 13,238×g, but for longer time periods, e.g., at least 4 h, preferably overnight. Using ultracentrifugation, it is possible to concentrate the virus titer of the composition of the invention by at least 10 fold, by at least 20 fold, by at least 30 fold, by at least 40 fold, by at least 50 fold, by at least 60 fold, by at least 70 fold, by at least 80 fold, by at least 90 fold, by at least 100 fold, by at least 150 fold, by at least 200 fold, by at least 250 fold, or even by at least 300 fold. However, even though ultracentrifugation has proven highly effective for harvesting lentiviral vectors, the method is associated with certain draw-backs: The rotors that may be used at such speed usually have only a small volume capacity and the attainment of large volumes of high-titer viral vectors thus is very time-consuming (Zhang et al., 2001). Ultracentrifugation further requires the virus particles to exhibit sufficient stability, e.g., due to pseudotyping of the viral envelope. Therefore, in another embodiment, the virus titer may also be concentrated by any other suitable purification method known from the state of the art, such as microfiltration, ultrafiltration, chromatography, density gradient ultracentrifugation, tangential flow microfiltration, or precipitation.

After concentrating the lentiviral particles of the invention by performing ultracentrifugation or any other suitable method from the state of the art, the viral particles have to be reconstituted in a suitable medium. The medium should have a physiological salt concentration and, preferably, a physiological pH. The inventors unexpectedly found that a defined mixture of phosphate-buffered saline (PBS) and 4-(2-hydroxyethyl)-1-piperazineethanesulfonic acid (HEPES) constituted a particularly beneficial medium for reconstituting and stabilizing virus particles. Therefore, the viral particles are preferably re-suspended in a medium comprising PBS (pH 7.2-7.4) and 1-50 mM, e.g., 1-10 mM, 20-30 mM, 30-40 mM, 40-50 mM, or, preferably, 10-20 mM HEPES. The present inventors found the addition of HEPES to be advantageous for maintaining the stability of the lentiviral particles of the invention, even after repeated Freeze-Thaw circles. Preferably, the medium used for reconstituting the concentrated viral particles does not contain any serum, hormones or cytokines to avoid any external biological stimulus on inner ear cells. Optionally, the medium may also be serum or blood. It may also be a lymphatic liquid of the inner ear, e.g., perilymph or endolymph.

The composition of the invention may be stored for up 1-4 weeks, e.g., 1 week, 2 weeks, 3 weeks or 4 weeks at temperatures around 4° C. Preferably, the composition is stored in a frozen state, most preferably at about −80° C. When frozen, the composition may be stored for at least 6, at least 12, at least 24, or at least 36 months prior to being administered to the subject.

Preferably, the cell of the inner ear that is to be transduced by the lentiviral vector according to the invention is a cell selected from the group consisting of cells of the organ of Corti, including inner and outer hair cells, spiral ganglion neurons and glial cells, cells of the stria vascularis and spiral ligament and lateral wall fibrocytes. Preferably, the cell is a hair cell or a spiral ganglion neuron. Hair cells constitute the sensory cells required for hearing and are located within the organ of Corti. They are arranged into one row of inner hair cells followed by three rows of outer hair cells. Hair cells possess apical modifications, so-called stereocilia, which are in contact with the fluid filling the scala media, the endolymph, and interdigitate with a variety of supporting cells. Unlike, e.g., birds or reptiles, mammals are incapable of naturally regenerating damaged or lost hair cells. Accordingly, hair cell degeneration may lead to permanent hearing loss if left untreated.

Spiral ganglion neurons (SGNs) are located within the modiolus, i.e., the central axis of the cochlea. Their dendrites form synapses with the base of hair cells and their axons run into the eighth cranial nerve, also known as the vestibulocochlear nerve. SGNs are the first neurons in the auditory systems that fire action potentials upon perception of sound. Glial cells are non-neuronal cells that do not produce electrical impulses and act as supporting cells of the nervous system. Glial cells have the potential to become reprogrammed into primary auditory neurons (PANs) through artificial expression of certain transcription factors (Meas et al., 2018, Reprogramming Glia Into Neurons in the Peripheral Auditory System as a Solution for Sensorineural Hearing Loss: Lessons From the Central Nervous System. Front Mol Neurosci. 11: 77). Glial cells thus represent an attractive target for lentiviral gene transfer to compensate for the loss of neurons in the peripheral auditory system. The stria vascularis consists of epithelial cells and forms the outer wall of the scala media. It furthermore produces the endolymph of the scala media. The spiral ligament secures the membranous scalar media to the spiral canal of the cochlea.

The inner ear cell transduced according to the invention may alternatively be type I and II vestibular hair cells, vestibular supporting cells and vestibular ganglion neurons. The vestibular system is the sensory system of the inner ear that facilitates the sense of balance and spatial orientation for coordinated movement. Accordingly, the composition according to the invention may also be suitable to alleviate symptoms associated with vestibular disturbances.

In another embodiment, the cell of the inner ear may also be a supporting cell selected from the group comprising a Boettcher cell, a Claudius cell, a Deiters' cell, a Hensen's cell or a pillar cell. Boettcher cells are located on the basilar membrane in the lower turn of the cochlea where they project microvilli into the intercellular space and may perform both secretory and absorptive functions. Claudius cells are located immediately above the Boettcher cells and are in direct contact with the endolymph. The formation of tight junctions between adjacent Claudius cells prevents the leakage of endolymph out of the scala media. Deiters' cells are arranged in up to 5 rows and sit directly on the basilar membrane of the cochlea. They form long, apical cell extensions that extend to the reticular lamina of the inner ear. The tall Hensen's cells are located adjacent to the outer row of Deiters' cells within the organ of Corti where they may mediate ion metabolism. Hensen's cells are a particularly interesting target for gene therapy as they are believed to have retained a capacity to regenerate hair cells (Malgrange et al., 2002, Epithelial supporting cells can differentiate into outer hair cells and Deiters' cells in the cultured organ of Corti. Cellular and Molecular Life Sciences. 59, 1744-1757). For example, a cargo nucleic acid may stimulate the regeneration of hair cells by Hensen's cells. Pillar cells can be divided in outer and inner pillar cells. Both types form numerous cross-linked microtubules and actin filaments. Pillar cells obtained their name because the outer cells are free standing and form contacts to adjacent cells only at their bases and apices.

The composition of the invention may be administered to the subject by any method suitable for delivering a substance to cells of the inner ear. The method for delivering the composition to a subject may, for instance, be a method selected from the group comprising a cochlea implant route, round window injection, oval window injection, canalostomy, cochleostomy as well as injection into the endolymphatic sac. For round window injection, a long needle is inserted through the outer ear and punctures the eardrum to inject the composition of the invention through the round window located in the medial wall of the middle ear into the scala tympani. Oval window injection involves injection of the composition through the oval window into the scala vestibuli. Alternatively, the composition may be injected into the endolymphatic sac, a small pouch connected via the endolymphatic duct to the endolymph-containing scala media and, thus, the compartment harboring the organ of Corti. However, injection into the endolymphatic sac requires a mastoidectomy that is associated with a large decompression of the posterior fossa plate. Thus, there is a risk associated with injection into the endolymphatic sac (Blanc et al., 2020). Cochleostomy is a method where the viral vector is injected directly into the cochlea. As previously described, direct injection into the cochlea harbors the risk of mechanically damaging sensory hair cells, leading to a decline in hearing function. During canalostomy, a hole is drilled into one of the semicircular canals of the vestibular organ and the composition is injected directly into the vestibular labyrinth. Mechanical damage to the cells of the inner ear, e.g., sensory hair cells, should be avoided in the context of the invention, especially in treatment of a human patient. Thus, the amount of the composition injected, as well as the pressure associated with the injection, should be minimized. Thus, a high titer and a high efficiency, which can be reached according to the invention, are of particular importance for treatment of humans.

The inventors could demonstrate that delivery of the composition into the mouse inner ear via canalostomy resulted in effective transduction of sensory hair cells and SGN. Therefore, if the subject according to the invention is a mouse or another rodent such as a guinea pig or a rat, the composition of the invention is preferably administered via canalostomy, in particular by injecting it into the posterior semicircular canal. Because a canalostomy does not disturb the middle ear during surgery and prevents injury of hair cells, it may also be suitable for vector delivery into the human inner ear (Blanc et al., 2020). Nevertheless, the method is associated with the risk of opening the membranous labyrinth and of a post-operative obstruction of the canal by fibrotic tissue. Improvements in the surgical techniques used, e.g., via the help of robotic assistance, may greatly reduce some of the risks associated with this method (Blanc et al., 2020). The surgical procedure conducted for canalostomy resembles that of a cochlear implant surgery (Blanc et al., 2020).

A cochlea implant is a device that is surgically introduced into the inner ear. Conventional cochlear implants consist of an external and an internal part. The external part is comprised of a microphone, a sound processor and a battery. The internal, transplanted part directs the signals received by the external part via an array of electrodes into the cochlea to directly stimulate the SGNs, thus circumventing the need for sensory hair cells to perceive sound. The implant may further be equipped with a reservoir chamber comprised of the composition of the invention as well as drug delivery channels that transport the composition from the reservoir chamber to the tips of the electrodes for targeted delivery into the cochlea. The implant may optionally be designed to allow for sustained release of the composition. Several types of cochlea implants and other auditory prostheses capable of delivering drugs to the inner ear have been developed and may be suitable for administering the composition of the invention to a subject (Hendricks et al., 2008, Localized Cell and Drug Delivery for Auditory Prostheses. Hear Res. 242(1-2), 117-131). Preferably, when the subject is a human, the composition is administered using a cochlear implant. In a further embodiment, the cochlear implant may also deliver a growth factor, e.g., a neurotrophic factor such as BDNF, GDNF, NT3 or IGF that may support, e.g., transdifferentiation of cells into sensory hair cells or SGNs.

The composition according to the invention may also be administered to the subject by other suitable methods, including known pump-catheter systems or round window membrane diffusion, the latter comprising the use of, e.g., a collagenase for partial digestion of the round window membrane. It is also possible that the composition of the invention is for use in administration to the subject via the systemic route. Intravenous injection of the composition under local anesthesia would constitute an atraumatic and easy alternative to the other methods discussed herein. Administration via the systemic route has already been successfully applied for delivery of AAVs into the murine inner ear (Shibata et al., 2017, Intravenous rAAV2/9 injection for murine cochlear gene delivery. Sci. Rep. 7, 9609). However, systemic delivery of the composition according to the invention would harbor the risk of off-target transduction and requires very high composition volumes and virus titers.

The inventors have shown that a high transduction efficiency or transduction rate can be obtained using the composition of the invention. Thus, preferably, the composition of the invention has a viral transduction efficiency in spiral ganglion neurons and hair cells of at least 10%, preferably, at least 30%. E.g., the transduction efficiency may be at least 35%, at least 40%, at least 50%, at least 55%, at least 60%, at least 65%.

The subject to be treated can be a human or non-human animal, preferably a vertebrate, e.g. a mammal such as a human, a primate, a dog, a cat, a rat, a guinea pig or a mouse. Preferably, the subject is a human being, e.g., a human having sensorineural hearing loss or in danger of developing sensorineural hearing loss.

In particular, the invention provides the following embodiments:
1. A composition for use in treating or preventing sensorineural hearing loss in a subject, wherein the composition has a titer of at least 10⁷ TU/mL and comprises a 3^{rd} generation lentiviral vector pseudotyped with a viral envelope glycoprotein capable of binding to a receptor expressed in a cell of the inner ear, wherein said composition is administered to the inner ear of the subject.
2. The composition for use of embodiment 1, wherein the lentiviral vector is pseudotyped with a viral envelope glycoprotein capable of binding to a receptor selected from the group consisting of the LDL-receptor and LDL-R family members, the SLC1A5-receptor, the Pit1/2-receptor and the PIRYV-G-receptor.
3. The composition for use of any of embodiments 1-2 wherein the lentiviral vector is pseudotyped with a viral envelope glycoprotein capable of binding the LDL-receptor or LDL-R family members, optionally, the LDL-receptor.
4. The composition for use of any of embodiments 1 or 3, wherein the viral envelope glycoprotein is selected from the group comprising MARAV-G, COCV-G, VSV-G and VSV-G ts.
5. The composition for use of embodiment 4, wherein the viral envelope glycoprotein is MARAV-G.
6. The composition for use of embodiment 4, wherein the viral envelope glycoprotein is COCV-G.
7. The composition for use of embodiment 6, wherein the viral envelope glycoprotein is VSV-G.
8. The composition for use of embodiment 4, wherein the viral envelope glycoprotein is VSV-G ts.
9. The composition for use of any of embodiments 1-2, wherein the lentiviral vector is pseudotyped with a viral envelope glycoprotein capable of binding the SLC1A5-receptor.
10. The composition for use of any of embodiments 1 or 9, wherein the viral envelope glycoprotein is selected from the group consisting of RD114 GP and derivatives thereof and BaEV GP and derivatives thereof.
11. The composition for use of embodiment 10, wherein the viral envelope glycoprotein is RD114 GP.
12. The composition for use of embodiment 10, wherein the viral envelope glycoprotein is derived from RD114 GP.
13. The composition for use of embodiment 10, wherein the viral envelope glycoprotein is BaEV GP.
14. The composition for use of embodiment 10, wherein the viral envelope glycoprotein is derived from BaEV GP.
15. The composition for use of any of embodiments 1-2, wherein the lentiviral vector is pseudotyped with a viral envelope glycoprotein capable of binding the Pit1/2-receptor.
16. The composition for use of any of embodiments 1 or 15, wherein the viral envelope glycoprotein is derived from a virus selected from the group consisting of GALV and 10A1 MLV.
17. The composition for use of embodiment 16, wherein the viral envelope glycoprotein is derived from GALV.
18. The composition for use of embodiment 16, wherein the viral envelope glycoprotein is derived from 10A1 MLV.
19. The composition for use of any of embodiments 1-2, wherein the lentiviral vector is pseudotyped with PIRYV-G capable of binding a PIRYV-G-receptor.
20. The composition for use of any of embodiments 1-19, wherein the lentiviral vector comprises a cargo sequence comprising a nucleic acid selected from the group comprising a protein-coding gene, a miRNA, an shRNA, a lncRNA and an sgRNA, wherein expression of said cargo sequence in said cell of the inner ear is capable of reducing, eliminating or preventing at least a symptom of sensorineural hearing loss in the subject.
21. The composition for use of embodiment 20, wherein the cargo sequence is a protein-coding gene, wherein a reduced or erroneous expression of said gene is associated with the development of the sensorineural hearing loss in the subject.
22. The composition for use of any of embodiments 20 or 21, wherein the cargo sequence is a protein-coding gene encoding a growth factor selected from the group comprising a neurotrophic factor such as BDNF, GDNF, NT3, IGF.
23. The composition for use of any of embodiments 20 to 22, wherein the cargo sequence is a protein-coding gene, wherein expression of said gene may compensate for a non-genetic cause of sensorineural hearing loss in the subject, optionally, by protecting inner ear cells from a hazardous agent or by increasing survival of inner ear cells that have been damaged by a hazardous agent, wherein the hazardous agent is selected from the group comprising noise and ototoxic drugs.
24. The composition for use of embodiment 20, wherein the cargo sequence is a miRNA, wherein a reduced or erroneous expression of said miRNA is associated with the development of the sensorineural hearing loss in the subject, or wherein said miRNA targets a gene whose elevated or erroneous expression is associated with the development of the sensorineural hearing loss in the subject.
25. The composition for use of embodiment 20, wherein the cargo sequence is an shRNA, wherein said shRNA targets a gene whose elevated or erroneous expression is associated with the development of the sensorineural hearing loss in the subject.
26. The composition for use of embodiment 20, wherein the cargo sequence is a lncRNA, wherein a reduced or erroneous expression of said lncRNA is associated with the development of the sensorineural hearing loss in the subject, or wherein said lncRNA regulates a gene whose elevated or erroneous expression is associated with the development of the sensorineural hearing loss in the subject.
27. The composition for use of embodiment 20, wherein the cargo sequence encodes a Cas9 protein and further comprises a single guide RNA for CRISPR/Cas9 delivery to a cell of the inner ear.
28. The composition for use of any of embodiments 1-27, wherein the hearing loss is caused by a mutation in a gene selected from the group comprising MYO7A, GJB2, OTOF, ATOH1, SLC26A4, TMC1, TMPRSS3, GIPC3, USH1C, RDX, CLDN14, WHRN, ESPN, ILDR1, SERPINB6, KCNQ4, POU4F3, SIX1, CCDC50, MIR96, POU3F4, EYA1, KCNQ1, GJB6, STRC, TECTA, GJB3, PRPS1, CDH23, GRXCR2, CABP2, GRXCR1, SYNE4, COL11A2, LRTOMT, BSND, PJVK, CIB2, CEACAM16, TMIE, NARS2, LHFPL5, MARVELD2, SLC22A4, KARS, SLC26A5, TPRN, ELMOD3, GPSM2, COCH, ESRRB, ADCY1, EPS8L2, CD164, OTOA, S1PR2, LOXHD1, TSPEAR, EPS8, CDC14A, MSRB3, DCDC2, PNPT1, TMEM132E, FAM65B, MYO3A, CLIC5, HGF, USH2A, TBC1D24, MET, PCDH15, PTPRQ, OTOGL, MYO6, TNC, TRIOBP, BDP1, MYO15A, DIAPH1, MYH14, DFNA5, WFS1, EYA4, COL11A2, MYH9, ACTG1, SLC17A8, GRHL2, DSPP, P2RX2, MYO1A, GFI1, TYMP and GFI1B.
29. The composition for use of any of embodiments 1-21, wherein the cargo sequence is a gene selected from the group comprising MYO7A, GJB2, OTOF ATOH1, SLC26A4, TMC1, TMPRSS3, GIPC3, USH1C, RDX, CLDN14, WHRN, ESPN, ILDR1, SERPINB6, KCNQ4, POU4F3, SIX1, CCDC50, MIR96, POU3F4, EYA1, KCNQ1, GJB6, STRC, TECTA, GJB3, PRPS1, CDH23, GRXCR2, CABP2, GRXCR1, SYNE4, COL11A2, LRTOMT, BSND, PJVK, CIB2, CEACAM16, TMIE, NARS2, LHFPL5, MARVELD2, SLC22A4, KARS, SLC26A5, TPRN, ELMOD3, GPSM2, COCH, ESRRB, ADCY1, EPS8L2, CD164, OTOA, S1PR2, LOXHD1, TSPEAR, EPS8, CDC14A, MSRB3, DCDC2, PNPT1, TMEM132E, FAM65B, MYO3A, CLIC5, HGF, USH2A, TBC1D24, MET, PCDH15, PTPRQ, OTOGL, MYO6, TNC, TRIOBP, BDP1, MYO15A, DIAPH1, MYH14, DFNA5, WFS1, EYA4, COL11A2, MYH9, ACTG1, SLC17A8, GRHL2, DSPP, P2RX2, MYO1A, GFI1, TYMP, GFI1B, BDNF, GDNF, NT-3, IGF1, IGF2, CNTF, ARTN, NRTN, ATP7B, MRP1, MRP2 and MDR1.
30. The composition for use of any of embodiments 1-29, wherein the lentiviral vector is derived from a virus selected from the group comprising HIV-1, HIV-2, SIV, FIV or EIAV.
31. The composition for use of embodiment 30, wherein the lentiviral vector is derived from HIV-1.
32. The composition for use of embodiment 30, wherein the lentiviral vector is derived from HIV-2.
33. The composition for use of embodiment 30, wherein the lentiviral vector is derived from SIV.
34. The composition for use of embodiment 30, wherein the lentiviral vector is derived from FIV.
35. The composition for use of embodiment 30, wherein the lentiviral vector is derived from EIAV.
36. The composition for use of any of embodiments 1-35, wherein the composition is obtainable from packaging the lentiviral vector in a process comprising providing at least four distinct expression plasmids.
37. The composition for use of embodiment 36, obtainable from a method wherein the at least four distinct expression plasmids are co-transfected into a packaging cell to produce the lentiviral vector particles, wherein the packaging cell is selected from the group comprising a HEK293T cell, a HEK293 cell, an NIH3T3 cell, a HeLa cell, an HT1080 cell, a COS-1 cell and an AGE1.CR cell to produce the lentiviral vector, preferably, a human cell line.
38. The composition for use of any of embodiments 36 or 37, wherein the ratio of the Gag/Pol packaging construct, the Env plasmid, the Rev plasmid and the vector plasmid is 7-16:1-7:3-8:4-10.
39. The composition for use of any of embodiments 1-38, wherein the lentiviral vector is replication incompetent due to the split packaging design and self-inactivating due to a deletion in the U3 region of the 3′ LTR.
40. The composition for use of any of embodiments 1-39, wherein the lentiviral vector lacks the genes vif, vpr, vpu, nef, and, optionally, tat.
41. The composition for use of any of embodiments 1-40, wherein the lentiviral vector comprises
   a) a 5′ LTR comprising a constitutively active heterologous promoter at the U3 position, a repeat region (R) and a U5 region,
   b) a 5′ UTR comprising a primer binding site (PBS), a splice donor site (SD), a packaging signal (ψ), a Rev-responsive element and, optionally, a splice acceptor (SA) site,
   c) an internal enhancer/promoter region operably linked to a cargo sequence,
   d) RNA processing elements optionally comprising a Woodchuck hepatitis virus post-transcriptional regulatory element (PRE), and
   e) a 3′ LTR with a deleted (SIN) U3 region, a repeat region (R) and a U5 region.
42. The composition for use of embodiment 41, wherein the heterologous promoter at the U3 position of the 5′ LTR is selected from the group comprising an RSV promoter, a CMV promoter, an HIV promoter, a eukaryotic promoter and a Tet-regulated promoter.
43. The composition for use of embodiment 41, wherein the internal enhancer/promoter region is selected from the group comprising a viral promoter, a cellular promoter, a cell-specific promoter, an inducible promoter, a hybrid promoter, and a synthetic promoter.
44. The composition for use of any of embodiments 41 or 43, wherein the internal enhancer/promoter region is a viral promoter selected from the group comprising SFFV, MPSV, MND, MESV, CMV, MLV and SV40.
45. The composition for use of any of embodiments 41 or 43, wherein the internal enhancer/promoter region is a cellular promoter selected from the group comprising EF1a, PGK1 and UBC.
46. The composition for use of any of embodiments 41 or 43, wherein the internal enhancer/promoter region is a hair cell-specific promoter and/or enhancer selected from the group comprising POU4F3, POU3F4, ATOH1, Prestin, Pendrin and MYO7A.
47. The composition for use of any of embodiments 41 or 43, wherein the internal enhancer/promoter region is an inducible promoter selected from the group comprising a TET-inducible promoter, a Cumate-inducible promoter, an electrosensitive promoter and an optogenetic switch.
48. The composition for use of any of embodiments 41 or 43, wherein the internal enhancer/promoter region is the hybrid promoter CAG.
49. The composition for use of any of embodiments 41 or 43, wherein the internal enhancer/promoter region is a synthetic promoter.
50. The composition for use of embodiment 41, wherein the internal enhancer/promoter region comprises a chromatin opening element.
51. The composition for use of any of embodiments 1-50, wherein the lentiviral vector is a non-integrating lentiviral vector.
52. The composition for use of any of embodiments 1-51, wherein the cell of the inner ear is a cell selected from the group consisting of cells of the organ of Corti, including supporting cells as well as inner and outer hair cells, spiral ganglion neurons and glial cells, cells of the stria vascularis and spiral ligament, lateral wall fibrocytes, type I and II vestibular hair cells, vestibular supporting cells and vestibular ganglion neurons.
53. The composition for use of embodiment 52, wherein the cell of the inner ear is a hair cell.
54. The composition for use of embodiment 52, wherein the cell of the inner ear is a spiral ganglion neuron.
55. The composition for use of embodiment 52, wherein the cell of the inner ear is a glial cell.
56. The composition for use of embodiment 52, wherein the cell of the inner ear is a cell of the stria vascularis.
57. The composition for use of embodiment 52, wherein the cell of the inner ear is a cell of the spiral ligament.
58. The composition for use of embodiment 52, wherein the cell of the inner ear is a lateral wall fibrocyte.
59. The composition for use of embodiment 52, wherein the cell of the inner ear is a type I or II vestibular hair cell.
60. The composition for use of embodiment 52, wherein the cell of the inner ear is a vestibular supporting cell.
61. The composition for use of embodiment 52, wherein the cell of the inner ear is a vestibular ganglion neuron.
62. The composition for use of any of embodiments 1-51, wherein the cell of the inner ear is a supporting cell selected from the group comprising a Boettcher cell, a Claudius cell, a Deiters' cell, a Hensen's cell or a pillar cell.
63. The composition for use of embodiment 62 wherein the cell of the inner ear is a Boettcher cell.
64. The composition for use of embodiment 62, wherein the cell of the inner ear is a Claudius cell.
65. The composition for use of embodiment 62, wherein the cell of the inner ear is a Deiters' cell.
66. The composition for use of embodiment 62, wherein the cell of the inner ear is a Hensen's cell.
67. The composition for use of embodiment 62, wherein the cell of the inner ear is a pillar cell.
68. The composition for use of any of embodiments 1-67, wherein the lentiviral vector titer is concentrated using ultracentifugation or other purification methods, incl. microfiltration, ultrafiltration, chromatography, density gradient ultracentrifugation, tangential flow microfiltration, or precipitation.
69. The composition for use of any of embodiments 1-68 that comprises a buffer having a physiological salt concentration and pH selected from the group comprising PBS with 10-20 mM HEPES.
70. The composition for use of any of embodiments 1-69, wherein the pharmaceutical composition is administered to the subject by a method selected from the group comprising a cochlea implant route, round window injection, oval window injection, canalostomy, cochleostomy as well as injection into the endolymphatic sac.
71. The composition for use of any of embodiments 1-70, wherein the composition is delivered to cells of the inner ear by injection into one of the semicircular canals of the vestibular labyrinth (canalostomy), wherein, preferably, the subject is a mouse.
72. The composition for use of any of embodiments 1-71, wherein the viral transduction efficiency in spiral ganglion neurons is at least 10%, optionally, at least 30%.
73. The composition for use of any of embodiments 1-72, wherein the subject is a human, a primate, a dog, a cat, a rat, a guinea pig or a mouse.
74. The composition for use of any of embodiments 1-73, wherein the subject is a human.
75. The composition for use of any of embodiments 1-74 which is for treating sensorineural hearing loss in a subject.
76. The composition for use of any of embodiments 1-74 which is for preventing sensorineural hearing loss in a subject.
77. A method for treating sensorineural hearing loss in a subject in need thereof, comprising the step of administering an effective amount of the composition of any of embodiments 1-76 to said subject.
78. A method for preventing sensorineural hearing loss in a subject in need thereof, comprising the step of administering an effective amount of the composition of any of embodiments 1-76 to said subject.

In summary, the invention teaches a composition comprising a third generation lentiviral vector for use in treating sensorineural hearing loss in a subject. The inventors were able, for the first time, to transduce sensory hair cells and SGNs in the inner ear of mice with efficiencies sufficiently high enough to result in correction of inner ear function or protection from progressive loss of inner ear function by combining an up-to-date lentiviral vector generation with an optimized production protocol and the reconstitution of viral vector in a defined-particle stabilizing medium. In doing so, the present invention demonstrates that lentiviral vectors have the potential to be successfully applied in gene therapy to treat sensorineural hearing loss in human patients.

Throughout the invention, the term “about” is intended to be understood as “+/−10%”. If “about” relates to a range, it refers to both lower and upper limits of the range. “A” is intended to mean “one or more”, if not explicitly mentioned otherwise.

All literature cited herein is herewith fully incorporated. The present invention is further illustrated, but not limited, by the following examples.

### BRIEF DESCRIPTION OF THE DRAWINGS

FIG. 1 Third-generation lentiviral vector (LV) system with split-packaging design and self-inactivating vector architecture (A) Schematic depiction of the LV architecture. SIN: self-inactivating; LTR: long terminal repeat, ΔU3: unique 3′ region with self-inactivating deletion; R: repeat region; U5: unique 5′ region; PBS: primer binding site; RRE: Rev-response element; cPPT: central polypurine tract; Prom.: promoter; GOI: gene of interest; PPT: polypurine tract; PRE: post-transcriptional regulatory element. (B) Four expression plasmids to provide the genomic vector information and all required structural and enzymatic (Gag-Pol, Env and Rev) components are transiently co-transfected into packaging cells, e.g., 293T cells, to produce vector particles.

FIG. 2 Efficient transduction of the HEI-OC1 and SV-k1 cell lines with LV pseudotyped with VSV-G. (A, B) HEI-OC1 (a cochlear HC-like conditionally transformed cell line) were transduced with LV preparations encoding for a dTomato reporter protein and analyzed by flow cytometry. (A) Eight different multiplicities of infection (MOI) were tested in HEI-OC1 cells. Analyses were performed on day 6 post-transduction. n=3 independent vector batches. Bars represent the mean transduction efficiencies. (B) Expression kinetics analysis in HEI-OC1 cells. A great proportion of the successfully transduced, reporter-positive cells was stably maintained until the final time point of analysis (56 days). n=2 independent vector batches. (C) Eight different multiplicities of infection (MOI) were tested in the stria vascularis cell line SV-k1. Analyses were performed on day 6 post-transduction. n=3 independent vector batches. Bars represent the mean transduction efficiencies. (D) Expression kinetics analysis in SV-k1 cells. A great proportion of the successfully transduced, reporter-positive cells was stably maintained until the final time point of analysis (56 days). n=2 independent vector batches (triangles and squares indicate separate batches). NTC, non-transduced control.

FIG. 3 Selected envelope glycoproteins efficiently pseudotype LV. Multiple batches were produced with each pseudotype to package a dTomato-reporter-encoding LV. Titers were assessed as the number of particles present per milliliter of supernatant by RNA extraction followed by qRT-PCR. Symbols indicate different batches. Bars represent the mean numbers of particles per ml. IND, Indiana strain; ts, temperature stable and complement resistant mutant; NJ, New Jersey strain.

FIG. 4 LV encoding a dTomato reporter protein and pseudotyped with different envelope proteins transduce the auditory cell line HEI-OC1 with different efficiency. Analysis was carried out by flow cytometry to determine (A) the percentage of dTomato-positive cells as well as the mean fluorescence intensity (MFI) of dTomato within the population of dTomato-positive cells. (B) Calculation of the relative expression of dTomato combines the two relevant parameters transduction efficiency (percentage of dTomato-positive cells) and expression level (MFI) as a correlate of the number of integrated vector copies per cell.

FIG. 5 The pseudotypes VSV-G, VSV-G ts, VSV-G^{NJ}, COCV-G and MARAV-G use the LDL-receptor (LDL-R) for cell entry while PIRYV-G-mediated cell entry does not involve LDL-R. Soluble LDL-R protein (sLDLR, 0.5 or 2.5/5 μg/ml) was added to HEI-OC1 cells prior to transduction with a LV encoding a dTomato reporter protein, packaged with different pseudotypes. Cell entry of the vector particles in the presence or absence of soluble LDL-R was monitored by flow-cytometry to determine the percentage of dTomato-positive cells. Values were normalized to the respective “no sLDLR” control. IND, Indiana strain; ts, temperature stable and complement resistant mutant; NJ, New Jersey strain.

FIG. 6 Efficient transduction of dissociated primary SGN with LV preparations pseudotyped with VSV-G. (A) Rat SGN were extracted, dissociated, seeded in in-vitro cultures and transduced at different multiplicities of infection (MOI) with a LV carrying an EGFP marker gene. Flow cytometry revealed efficient transduction, with a low MOI of 1 already yielding more than 20% of EGFP-positive cells. (B) SGN culture transduced with a LV expressing an EGFP reporter. Top: brightfield; bottom: EGFP channel. (C) Immunocytochemistry of SGN transduced with a LV expressing a dTomato reporter gene. Neurofilament: marker for SGN.

FIG. 7 LV encoding a dTomato reporter protein and pseudotyped with different envelope proteins transduce primary dissociated SGN cultures with different efficiency. Analysis was carried out by flow cytometry to determine (A) the percentage of dTomato-positive cells as well as the mean fluorescence intensity (MFI) of dTomato within the population of dTomato-positive cells and to calculate (B) the relative expression of dTomato, combining the two relevant parameters transduction efficiency (percentage of dTomato-positive cells) and expression level (MFI) as a correlate of the number of integrated vector copies per cell. (n=3 for each envelope protein used to pseudotype LV).

FIG. 8 Efficient transduction of primary organ of Corti explants with VSV-G pseudotyped LV preparations. Rat organs of Corti were explanted and cultured in vitro. Transduction of these cultures with (A) an EGFP-expressing or (B) a dTomato-expressing LV yielded a high transduction efficiency throughout the explant.

FIG. 9 Inner ear architecture and canalostomy. (A) The inner ear contains the labyrinth, including three semicircular canals, and the cochlea. Viral particles can be administered via canalostomy through a hole drilled into the posterior semicircular canal. (B) Cochlea cut in cross section. The organ of Corti (black box) sits on the basilar membrane, a membrane which separates the scala tympani from the scala media. (C) The organ of Corti contains inner and outer hair cells. Spiral ganglion neurons (SGN) project the generated electrical signal to the brain.

FIG. 10 Efficient transduction of SGN and HC upon in-vivo LV injection. A VSV-G pseudotyped LV encoding for a dTomato reporter protein was administered in vivo to adult mice through canalostomy. Animals were sacrificed on day 30. Epithelium, spiral ganglion neurons (SGN) and hair cells (HC) were highly positive for the marker protein. Red signal: dTomato; blue signal: DAPI. (A) Exemplary section of the cochlea. (B) Higher magnification of the section highlighted in A by a white box (the organ of Corti), to better identify inner (IHC) and outer (OHC) hair cells.

FIG. 11 LV vector particles delivering a dTomato reporter transgene efficiently transduce the murine inner ear. (A) VSV-G pseudotyped particles delivering a dTomato reporter, expressed under control of the CAGs promoter, were administered via canalostomy. The cochleae were removed at 2 months post-delivery, cleared and expression of the dTomato protein determined to characterize the transduction pattern. Both inner and outer hair cells and SGN are dTomato-positive, indicating successful transduction. (B) Untreated control processed analogously to the cochleae of treated animals. Some false-positive cells are present in the stria vascularis.

FIG. 12 Efficient transduction of several different cell types within the inner ear upon in-vivo injection of LV encoding for a dTomato reporter protein and pseudotyped with VSVG^{IND}, VSV-G^{IND}ts, COCV-G, MARAV-G, Ampho glycoprotein or EboZ glycoprotein. Cochlear sections are shown. Indicated are transduced cells (red) of the stria vascularis (*), HCs (white arrows), neurites (#) and spiral ganglion cells (§). Cell nuclei are stained with DAPI (blue).

FIG. 13A third generation (SIN) LV with an internal SFFV promoter (Prom.) to express the therapeutically relevant codon-optimized human Claudin 14 (hCLDN14co) transgene and a dTomato reporter fluorescent protein produced high titers. (A) Schematic depiction of the vector to treat hearing loss due to loss of CLDN14. GOI, gene of interest. (B) Titration of VSV-G pseudotyped CLDN14 LV in HT1080 cells showed high titer production.

FIG. 14 Efficient and dose-dependent transduction of 293T cells with VSV-G pseudotyped LV designed to deliver human Claudin 14 (hCLDN14) and a dTomato fluorescent marker protein. (A) Transduction rates ranged between 50% and over 80% as determined by flow cytometric analysis of dTomato expression. Transduction rates were dependent on the multiplicity of infection (MOI) applied. (B and C) Immunoblot analyses showed successful expression of the dTomato fluorescent marker protein (B) and hCLDN14 (C). The protein expression levels correlated with the percentages of transduced cells, with higher protein levels detected in the cells transduced at higher MOI.

FIG. 15 Successful forced expression of hCLDN14 in cochlear explants from CLDN14 knock-out mice. Following transduction of cochlear explants with either a monocistronic LV designed to express the dTomato red fluorescent protein or a bicistronic LV designed to express hCLDN14 and dTomato, the dTomato red fluorescent marker protein was detected in cochlear explants. In contrast, dTomato expression was not observed in non-transduced control (NTC) explant cultures. Similarly, hCLDN14 expression was only detected in explants transduced with the VSV-G pseudotyped LV CLDN14 vector, as shown by anti-CLDN14 antibody staining (green). The scale bar represents 100 μm.

FIG. 16 LV particles delivering a human Myosin VIIa transgene can be successfully produced. (A) Schematic depiction of the vector to treat hearing loss due to Myosin VIIa deficiency. Expression of the human Myosin VIIa (hMYO7A) cDNA sequence is driven by either an SFFV or a CAGs promoter. For initial tests, the transgene is combined with a dTomato reporter via an IRES sequence (i2.dTomato) to facilitate identification of transduced cells. (B) Titer of VSV-G pseudotyped particles produced with the vector variants shown in A. Titers were determined based on flow cytometry of transduced cells, expressed as transducing units per milliliter (TU/mL), for preparations including the dTomato reporter, and based on qPCR quantifying the number of vector copies in transduced cells, expressed as infectious genomes per milliliter (IG/mL), for preparations devoid of the dTomato reporter.

FIG. 17 Lentiviral delivery of MYO7A results in survival of hair cells in a murine Myo7a knock-out (KO) hearing loss mouse model. Shaker-1 mice were injected with VSV-G pseudotyped LV particles delivering hMYO7A at day 21 of age. Cochleae were removed at 2 months of age. (A-C) Confocal image of phalloidin staining of actin in inner hair cells of midturn whole mount cochleae. (A) Shaker-1^{+/-} (heterozygous for Myosin VIIa KO); (B) Shaker-1^{-/- (homozygous KO), untreated: a complete loss of hair cells is seen; (C) Shaker-}1^{-/- (homozygous KO), lentivirally treated: survival of hair cells upon LV therapy.}

FIG. 18 LV MYO7A gene therapy improves balance function in homozygous Shaker-1 mice. Homozygous mutant shaker mice, which are deficient in Myo7a expression, were treated with a third-generation VSV-G pseudotyped LV expressing MYO7A and a dTomato tag delivered via the posterior semicircular canal at age P17. Prior evaluation suggests at this age there is a targetable population of hair cells and some residual hearing. (A) Evaluation of balance by rural testing showed that both at one and two months (1 mo, 2 mo) post-vector delivery there was a statistically significant maintenance of rotarod times compared to untreated controls. This however was statistically less than heterozygous littermates. (B) Evaluation using actimeter testing showed that there were no significant differences in total distance traveled between heterozygous (+/−) untreated controls and LV treated homozygous (−/−) littermates, showing significant improvement as compared to untreated homozygous (−/−) littermates. (C) Evaluation of total degrees circled showed that LV treated homozygous (−/−) animals had reduced circling behavior compared to untreated age matched littermates.

FIG. 19 LV MYO7A gene therapy arrests hearing loss progression. Heterozygous (+/−) Shaker-1 mice develop a significant hearing loss by 6 months of age compared to homozygous wild type (+/+) mice (P<0.001 at all frequencies). Shaker-1 mice were injected at postnatal day 4 with VSV-G pseudotyped LV particles delivering hMYO7A or left untreated. Auditory brainstem recordings (ABR) were performed to determine the threshold dB at different sound frequencies. Treatment with LV MYO7A prevented progression of hearing loss at all frequencies.

### EXAMPLE

In the following example, the preparation of pseudotyped LV and their use for effective transduction of sensory hair cells and spiral ganglion neurons are demonstrated.

### Materials and Methods

### HEK293T Cell Culture

### Materials

| | |
|---|---|
| Basic culture | Dulbecco's Modified |
| medium | Eagle's Medium (DMEM) |
| Fetal bovine | heat-inactivated (h.i.) for |
| serum (FBS) | 30 min @ 56° C. |
| Penicillin/Streptomycin | 10.000 U Penicillin; |
| (Pen/Strep) | 10 mg/ml Streptomycin |
| Sodium pyruvate | 100 mM |
| Phosphate-buffered | 1x |
| saline (PBS) | |
| Trypsin/EDTA [10x] | 0.5%/0.2% in PBS |
| Complete culture | DMEM + 10% (v/v) h.i. FBS + 1% (v/v) |
| medium | Pen/Strep + 1% (v/v) sodium pyruvate |

### Procedure

HEK293T cells are kept in complete culture medium at 37° C. in the presence of 5% CO₂
Cells are passaged every 2^{nd}-3^{rd} day
cells are rinsed with 1×PBS
cells are detached by treatment with 1× trypsin/EDTA and incubation for min @ 37° C.
the reaction is stopped with standard culture medium
the culture is split at the respective ratio (usually 1:8-1:12)

### Viral Vector Production

### Materials

| | |
|---|---|
| HEPES | 1M |
| Chloroquine | 25 mM |
| HeBS (2x) | Ultra-pure water, 50 mM HEPES, |
| | 280 mM NaCl, 1.5 mM Na₂HPO₄ |
| Calcium chloride (CaCl₂) | 2.5M |
| Lentiviral vector | according to the vector |
| transfer plasmid | to be packaged |
| Lentiviral Gag/Pol | pcDNA3.GP.4xCTE |
| expression plasmid | |
| Envelope glycoprotein | see list of Env GPS |
| expression plasmid | |
| Rev expression plasmid | pRSV-Rev |
| Filters | Millex-GP syringe filter (0.22 μm) |

| Envelope glycoprotein (Env GP) | Env GP packaging plasmid |
|---|---|
| VSV-G | pMD.G |
| VSV-G ts | CMV.VSVg_thermos.IRES.Puro |
| COCV-G | CMV.MPMV.ASLV.CocalV |
| MARAV-G | phCMV-Maraba-G |
| PIRYV-G | phCMV.PIRYV-G.iPuro 2 |
| Ampho | phCMV-A-MLVenv |
| BaEV | pcDNA3.BaEV |
| BaEV\TR | pcDNA3.BaEV-TR |
| RD114 | phCMV-RD114 |
| RD114\TR | phCMV-RD114-TR |

### Procedure

Day 0: Cell seeding
   HEK293T cells are seeded on tissue-culture-grade plastic dishes in standard culture medium
Day 1: Transient transfection of packaging components
   All packaging components are combined in sterile water with the Gag/Pol packaging construct, the Env plasmid, the Rev plasmid and the vector plasmid in a ratio of 13-16:1-7:6-8:6-8
   10% (v/v) [250 mM] calcium chloride are added and mixed well
   the DNA/CaCl₂) mixture is added dropwise to an equal volume of 2×HeBS while generating air bubbles with an electronic pipette filler, during addition of the DNA mix and for another 30 seconds
   the mixture is incubated for 15-20 minutes at room temperature
   meanwhile, the medium on the cultured cells is replaced by fresh complete culture medium supplemented with 1-2% (v/v) [10-20 mM] of HEPES and 0.1% (v/v) [25 μM] chloroquine
   after the 20 min incubation, the transfection mix is dropwise added to the cells
   the cells are cultured at 37° C. and 5% CO₂
   6-16 hours post-transfection, the medium is replaced by fresh complete culture medium supplemented with 10-20 mM HEPES
Day 3: Harvest I
   30-36 hours post-transfection, the medium containing produced lentiviral particles is harvested and passed through a 0.22 UM pore size filter
   fresh complete culture medium supplemented with 10-20 mM HEPES is to the transfected cells for a second harvest
Day 4: Harvest II
   48-54 hours post-transfection, the second harvest is accomplished as per the procedure of the first harvest
   the two harvests (I and II) from each plate are pooled
   the viral vector preparation is either immediately frozen at −80° C. without further concentration or concentrated using ultracentrifugation

### Viral Vector Concentration and Reconstitution

### Procedure

The viral vector preparation is loaded into SW28 polyallomer ultracentrifuge tubes
For concentration, the viral vector particles are spun at 4° C. at different speeds and runtimes, depending on the pseudotype (see table)

| Envelope glycoprotein (Env GP) | Env GP packaging plasmid |
|---|---|
| VSV-G | pMD.G |
| VSV-G ts | CMV.VSVg_thermos.IRES.Puro |
| COCV-G | CMV.MPMV.ASLV.CocalV |
| MARAV-G | phCMV-Maraba-G |
| PIRYV-G | phCMV.PIRYV-G.iPuro 2 |
| Ampho | phCMV-A-MLVenv |
| BaEV | pcDNA3.BaEV |
| BaEV/TR | pcDNA3.BaEV-TR |
| RD114 | phCMV-RD114 |
| RD114/TR | phCMV-RD114-TR |

The supernatant is decanted off and the pellet containing the viral particles is resuspended in reconstitution buffer consisting of PBS supplemented with 1-2%1 M HEPES
The volume used for resuspension is usually chosen to concentrate the vector preparation 50-300 fold
Aliquots are prepared and stored at −80° C. until further usage

Table 1 presents a collection of different LV particles harboring a variety of therapeutic transgenes as a cargo that have been successfully produced using the herein described method. The table further provides information on the promoters used for controlling the expression of the respective transgenes, co-expressed reporter proteins as well as the titers yielded for each of the LV particle preparations.

TABLE 1
Successful production of LV particles to transfer a therapeutic gene

| | | Co- | | |
|---|---|---|---|---|
| Therapeutic gene/ | | expressed | LV | Titer |
| combination | Promoter | reporter | titer | Unit |
| hMYO7A | SFFV | — | 2.75E+07 | IG/mL |
| hMYO7A | SFFV | IRES.dTomato | 2.84E+07 | IG/mL |
| hMYO7A | CAGs | — | 1.88E+07 | IG/mL |
| hMYO7A | CAGs | IRES.dTomato | 7.78E+06 | IG/mL |
| hMYO7A | SFFV | IRES.dTomato | 8.66E+05 | TU/mL |
| hMYO7A | CAGs | IRES.dTomato | 9.07E+05 | TU/mL |
| hMYO7A | SFFV | IRES.dTomato | 9.83E+05 | TU/mL |
| hMYO7A | CAGs | IRES.dTomato | 1.58E+06 | TU/mL |
| hCLDN14 | SFFV | IRES.dTomato | 3.52E+07 | TU/mL |
| hCLDN14 | SFFV | IRES.dTomato | 1.55E+08 | TU/mL |
| hCLDN14 | SFFV | IRES.dTomato | 2.57E+08 | TU/mL |
| hGDNF | SFFV | IRES.dTomato | 2.33E+08 | TU/mL |
| ARTN | SFFV | IRES.dTomato | 1.66E+08 | TU/mL |
| ARTN | SFFV | IRES.dTomato | 4.66E+08 | TU/mL |
| SLC26A5 | SFFV | — | >1E+08 | TU/mL |
| SLC26A5 | SFFV | IRES.EGFP | >1E+08 | TU/mL |
| GjB2 | SFFV | IRES.dTomato | 4.23E+08 | TU/mL |
| hBDNF | SFFV | IRES.dTomato | 3.09E+08 | TU/mL |
| hBDNF | CAG | IRES.dTomato | 1.06E+07 | TU/mL |
| hBDNF | CMV | IRES.dTomato | 1.34E+08 | TU/mL |
| NRTN | SFFV | IRES.dTomato | 6.00E+07 | TU/mL |
| NTF3 | SFFV | IRES.dTomato | 4.38E+08 | TU/mL |
| IGF1/HOXA9 | SFFV | IRES.dTomato | 4.59E+08 | TU/mL |
| IGF2/HOXA9 | SFFV | IRES.dTomato | 4.77E+08 | TU/mL |
| POU4F3 | SFFV | IRES.dTomato | 1.20E+08 | TU/mL |
| POU3F4 | SFFV | IRES.dTomato | >1E+08 | TU/mL |
| ATOH1 | SFFV | IRES.EGFP | 4.02E+08 | TU/mL |
| GFI1 | SFFV | IRES.dTomato | >1E+08 | TU/mL |
| SOX2 | SFFV | IRES.EGFP | 2.98E+08 | TU/mL |
| NOTCH1 ICD | SFFV | IRES.dTomato | >1E+08 | TU/mL |
| miR96 | SFFV | EGFP | 6.00E+08 | TU/mL |
| EYA1 | SFFV | IRES.EGFP | 4.68E+08 | TU/mL |
| EYA1/SOX2 | SFFV | IRES.EGFP | 2.79E+08 | TU/mL |
| WFS1 | SFFV | IRES.dTomato | 4.86E+08 | TU/mL |
| | CAGs | GFP | 1.89E+09 | TU/mL |
| | CAGs | idTomato | 1.56E+09 | TU/mL |
| NEUROG1 | SFFV | IRES.EGFP | 6.60E+08 | TU/mL |
| ATOH1/POU4F3/ | SFFV | IRES.EGFP | 1.42E+08 | TU/mL |
| SOX2 | | | | |
| EYA1/SOX2/SIX1 | SFFV | IRES.EGFP | 2.43E+08 | TU/mL |
| ATOH1/POU4F3/ | SFFV | IRES.EGFP | 2.79E+08 | TU/mL |
| GFI1 | | | | |

IRES, internal ribosomal entry site; TU, transducing units; IG, infectious genomes.

### In Vivo Application of Lentiviral Vector Particles

### Procedure

Adult mice are anesthetized with an intraperitoneal (IP) injection of a mixture of ketamine (150 mg/kg), xylocaine (6 mg/kg) and acepromazine (2 mg/kg) in sodium chloride 0.9%. A dorsal postauricular incision is made, and the posterior semicircular canal exposed. Using a microdrill, a canalostomy is created, exposing the perilymphatic space. Subsequently, 1 μL of vector is injected using a Hamilton microsyringe with 0.1 μL graduations and a 36 gauge needle. The canalostomy is sealed with bone wax, and the animals are allowed to recover for 72 hours. The mice showed no signs of vestibular dysfunction or head tilt postoperatively.

### Potential Human Delivery:

Three main routes can be considered for delivery into the inner ear in humans. Vector can be delivered through the stapes footplate using standard stapedectomy approaches. This delivers to the vestibular organ and scala vestibuli. After elevating the tympanic membrane, the stapes footplate is visualized. Using a CO₂ laser, a 250 μm hole is created in the footplate. An infusion catheter is placed into the stapedotomy not exceeding a depth of 0.5 mm. Infusion of vector at speeds not exceeding 10 μL/min is carried out.

Injections into the round window can be accomplished using middle ear based or posterior tympanotomy approaches. This allows delivery into the scala tympani. For the middle ear approach, the tympanic membrane is elevated, and the bony round window overhang removed with a microdrill. The round window pseudo membrane is removed. At this point, the round window can be injected. For the round window, microinjection devices as well as a cochlear catheter developed by MedEI GmbH may be used.

Finally, in humans, delivery could also be carried out into the scala media by injecting into the endolymphatic sac. After completing a mastoidectomy, the facial nerve and posterior semicircular canal are identified. The endolymphatic sac is identified and can then be accessed. All of these approaches are based on standardized ontological surgical procedures and use standard operating room equipment and anaesthesia.

### Results

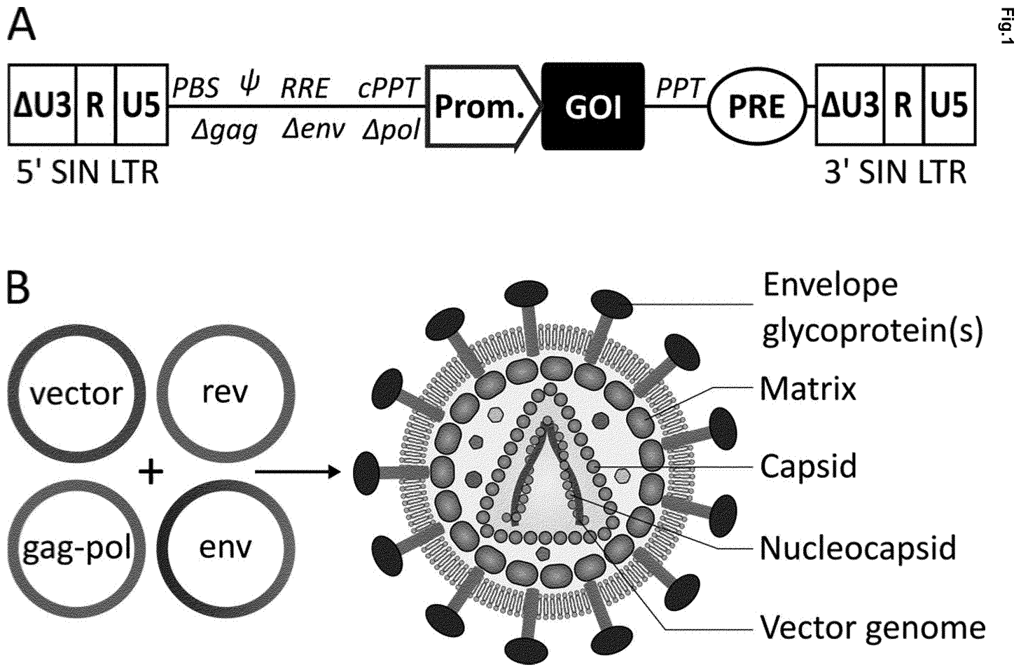

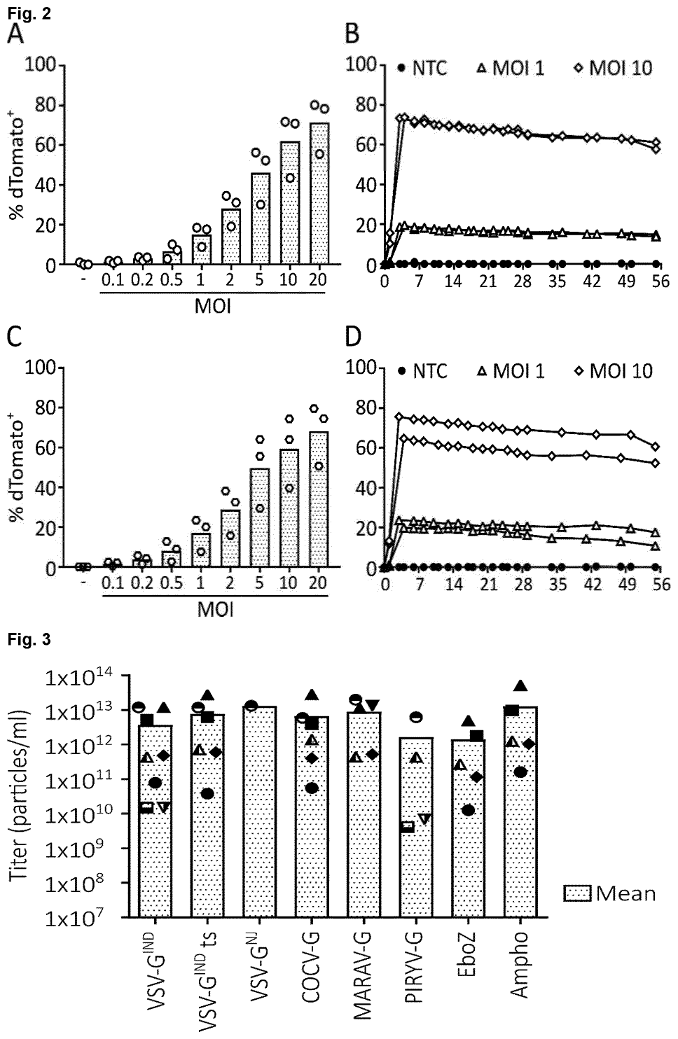

The improved LV system illustrated in FIG. 1 was used to efficiently transduce different otic cell types, for example the cochlear HC-like cell line HEI-OC1 and the stria vascularis cell line SV-k1 (FIG. 2). Transduction efficiencies of up to 70%, e.g., over 20%, over 40% or over 60%, were achieved with low MOI (multiplicities of infection) and transgene expression was stable over at least 56 days (FIG. 2).

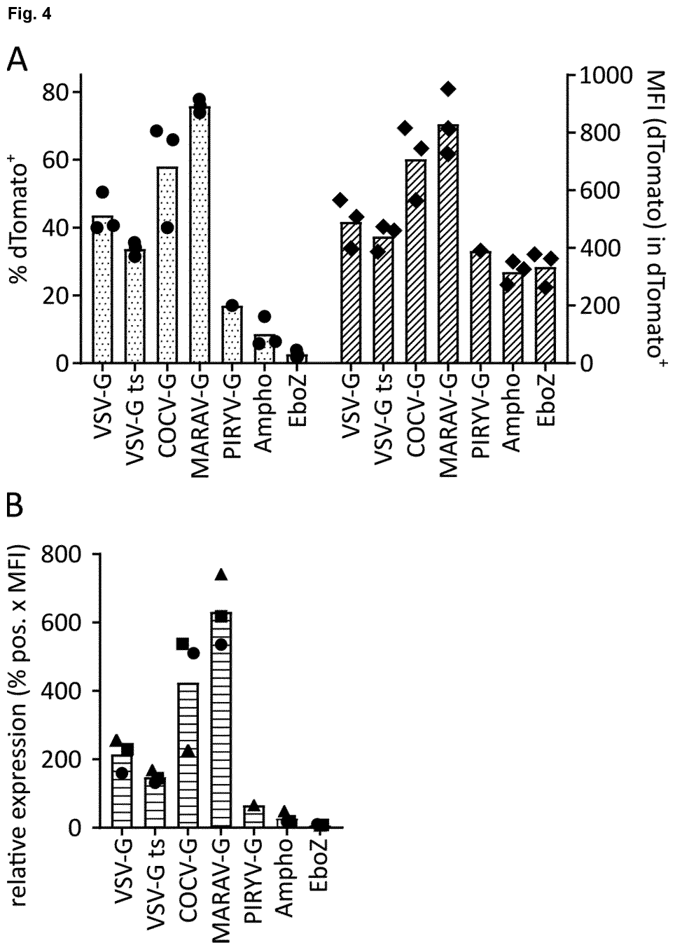

LV particles can be efficiently pseudotyped with different envelope glycoproteins, including VSV-G^{IND}, VSV-G^{IND} ts, VSV-G^{NJ}, COCV-G, MARAV-G, PIRYV-G, EboZ and Ampho (FIG. 3). Using the dTomato fluorescence protein as a reporter for cell transduction efficiency, LV particles pseudotyped with the above named envelope glycoproteins were directly compared in HEI-OC1 cells. Flow cytometric analyses showed that LV particles pseudotyped with different envelope glycoproteins differed in their transduction efficiencies, in descending order of cell transduction efficiency (% dTomato expressing cells) and transgene expression level (MFI of dTomato in dTomato+ cells): MARAV-G, COCV-G, VSV-G, VSV-Gts, PIRYV-G, Ampho and EboZ (FIG. 4).

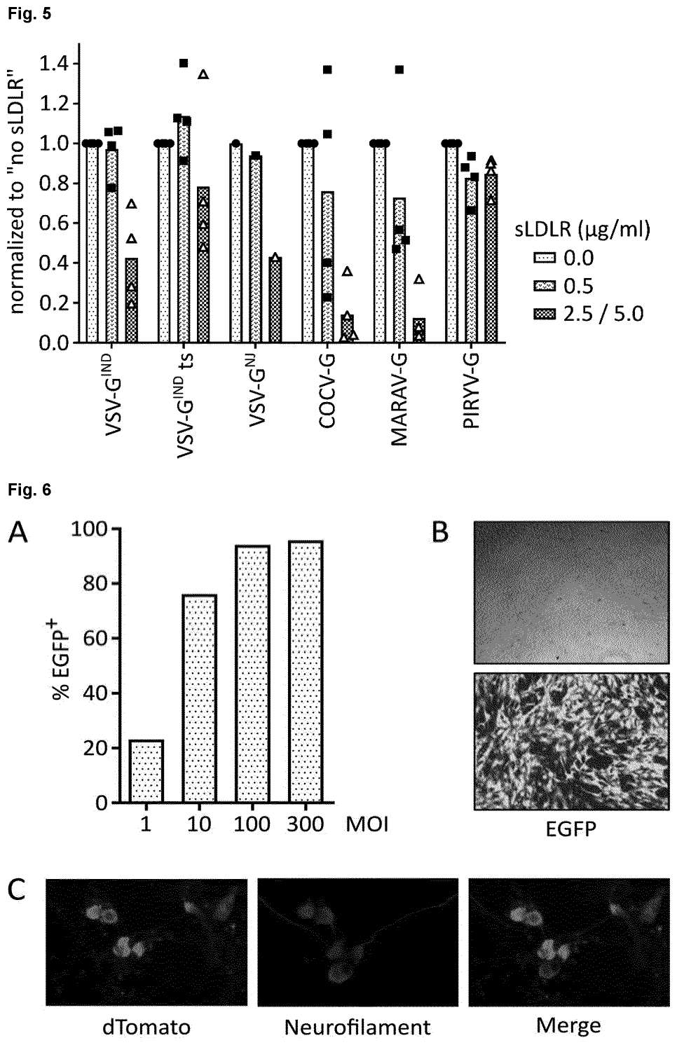

Cell entry blocking experiments demonstrated that LV particles pseudotyped with VSV-G^{IND}, VSV-G^{IND} ts, VSV-G^{NJ}, COCV-G and MARAV-G use the LDL-receptor (LDL-R) for cell entry while PIRYV-G-mediated cell entry may not involve LDL-R (FIG. 5). The blocking was more pronounced for COCV-G and MARAV-G pseudotyped particles than for VSV-G counterparts (FIG. 5).

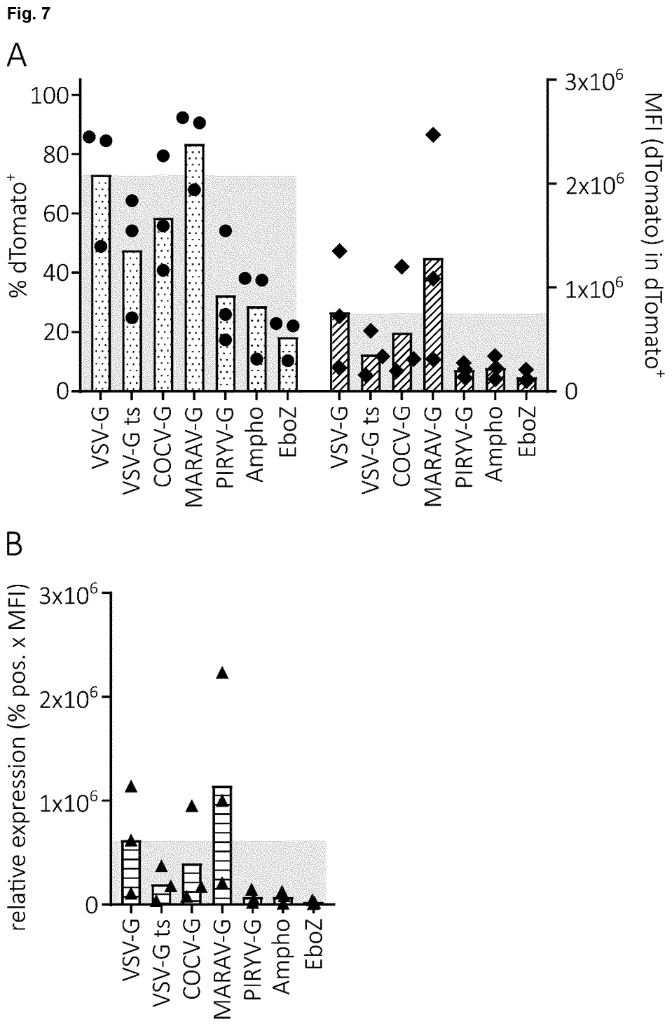

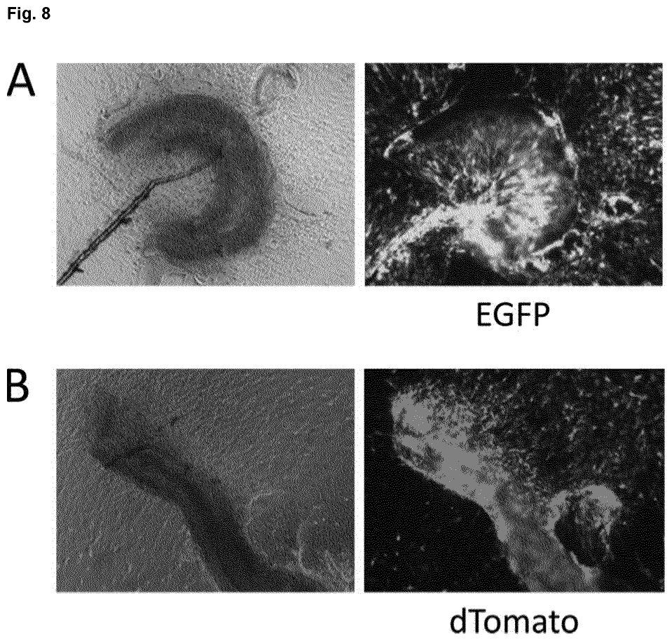

LV particles engineered to express EGFP and pseudotyped with the VSV-G envelope glycoprotein efficiently transduced primary rat SGN in vitro, with transduction of over 20% of SGN at low MOI and over 80% at higher MOI as demonstrated by flow cytometric analyses of EGFP (FIG. 6A). Furthermore, EGFP transgene expression in transduced SGN is clearly shown by immunofluorescence microscopy (FIG. 6B). Immunohistochemical staining for the SGN marker neurofilament showed successful transduction of SGN with a LV designed to express dTomato (FIG. 6C).

Similarly to the data shown above in FIG. 4, LV that encode the dTomato fluorescence protein as a reporter for cell transduction efficiency transduced primary SGN ex vivo cultures with different efficiency depending upon the envelope glycoprotein used to pseudotype the LV particles (FIG. 7). Flow cytometric analyses showed that LV particles pseudotyped with different envelope glycoproteins had good transduction efficiencies, except for those pseudotyped with the EboZ glycoprotein, but resulted in different levels of transgene expression, with highest transgene expression with MARAV-G pseudotyped particles, followed by those pseudotyped with VSV-G, COCV-G, VSV-Gts, PIRYV-G, Ampho and EboZ envelope glycoproteins (FIG. 7).

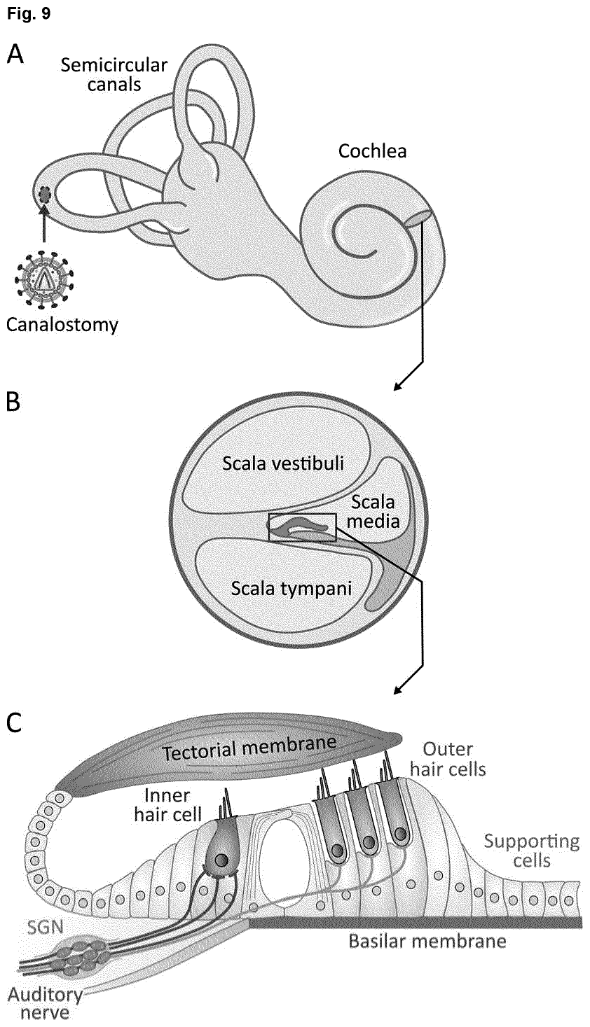

VSV-G pseudotyped LV containing either EGFP or dTomato as transgene efficiently transduced primary rat organ of Corti explants as shown by fluorescence microscopy photographs (FIG. 8). Note that EGFP and dTomato are strongly expressed throughout the respective explants (FIG. 8).

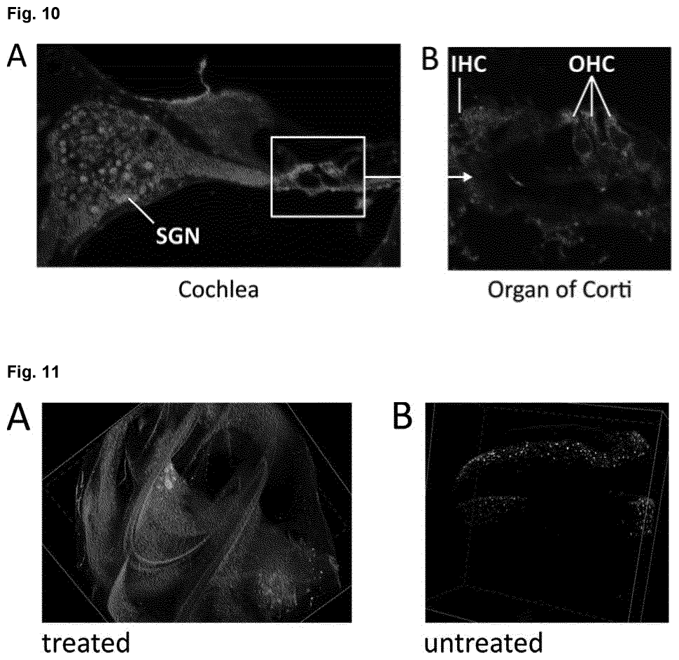

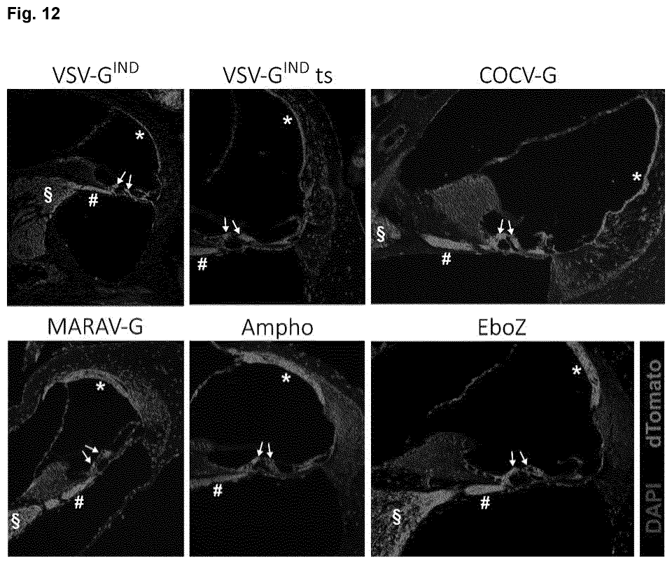

Efficient in vivo transduction of mouse SGN and HC was observed upon in vivo injection of a VSV-G pseudotyped LV encoding a dTomato reporter protein as transgene (FIG. 10). Murine inner ear cells throughout the entire cochlea (from base to apex), including IHC and OHC, were efficiently transduced in vivo with LV particles engineered to express a dTomato reporter protein (FIG. 11).

All different lentiviral pseudotypes included in this patent application successfully and efficiently transduced relevant cochlear cell types upon in vivo injection into normal hearing mice. Importantly, both outer and inner HC, as well as SGN and cells of the stria vascularis, were positive for the vector-encoded dTomato reporter protein (FIG. 12). LV pseudotyped with the MARAV-G, Ampho and EboZ glycoproteins appeared to transduce more layers of cells (i.e. also deeper layers) within the stria vascularis.

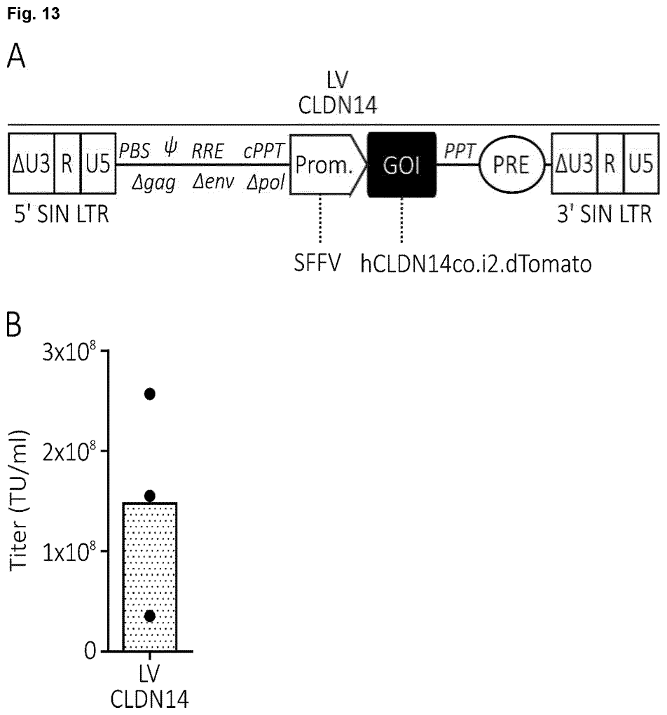

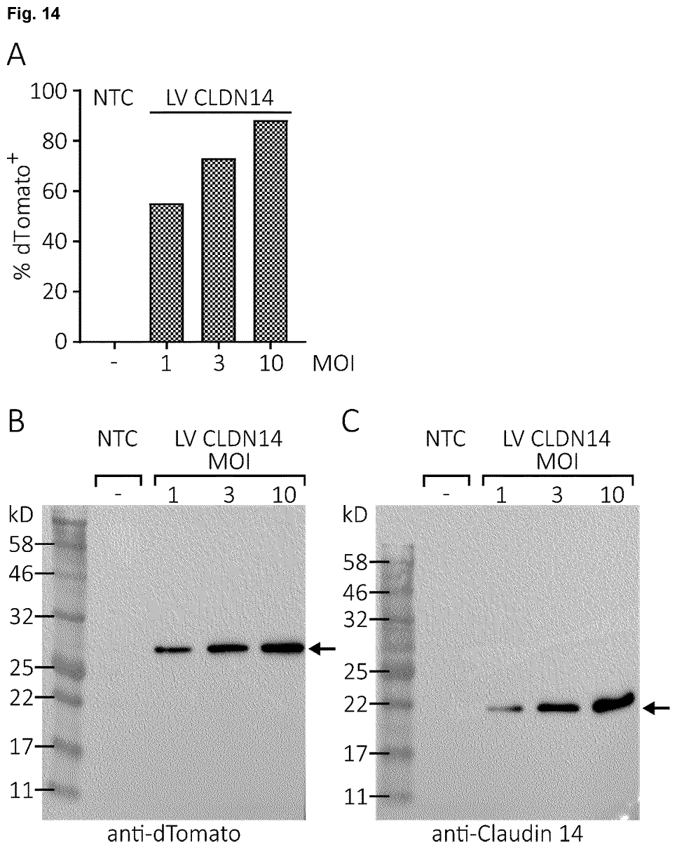

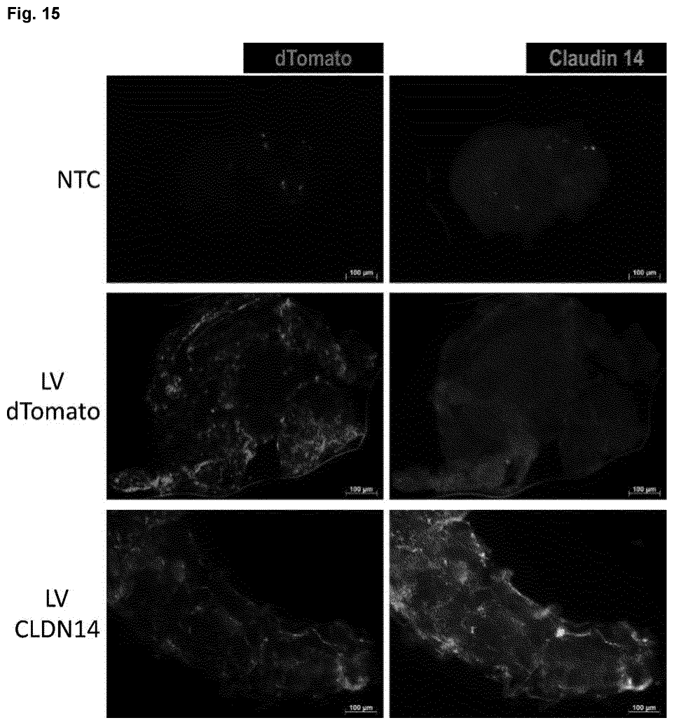

LV particles designed to deliver the human Claudin 14 (CLDN14) transgene to treat hearing loss due to Claudin 14 deficiency were successfully produced at high titers (FIG. 13). Transduction of 293T cells confirmed vector functionality, with dose-dependent expression of the encoded CLDN14 and reporter dTomato proteins (FIG. 14). Importantly, application of the lentiviral CLDN14 vector could achieve CLDN14 expression in primary explants from Cldn14 knockout mice (FIG. 15).

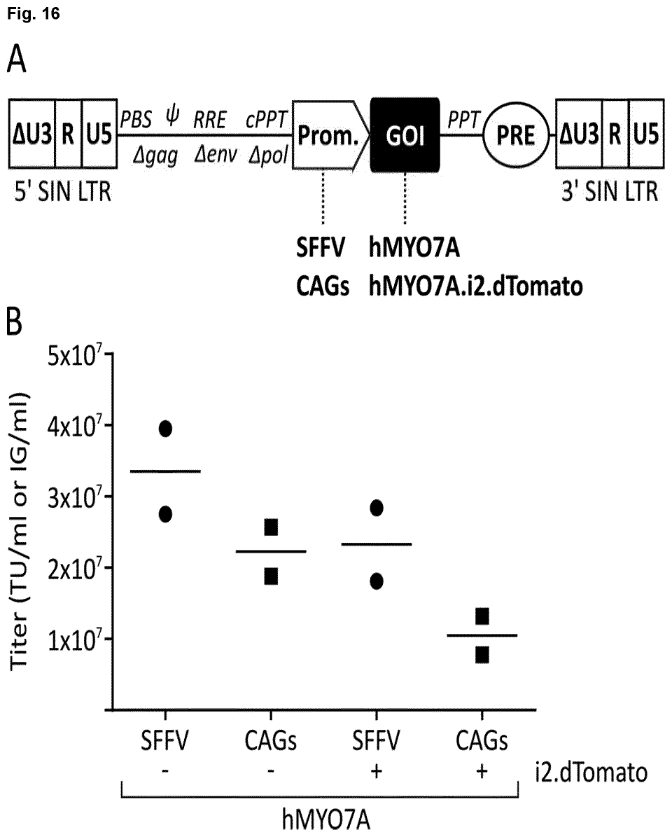

For a second therapeutically relevant setting, LV particles designed to deliver a human Myosin VIIa (MYO7A) transgene to treat hearing loss due to Myosin VIIa deficiency were successfully produced (FIG. 16). LV titers ranged from 1×10⁷-3×10⁷ TU/mL and 2×10⁷-4×10⁷ IG/mL (FIG. 16).

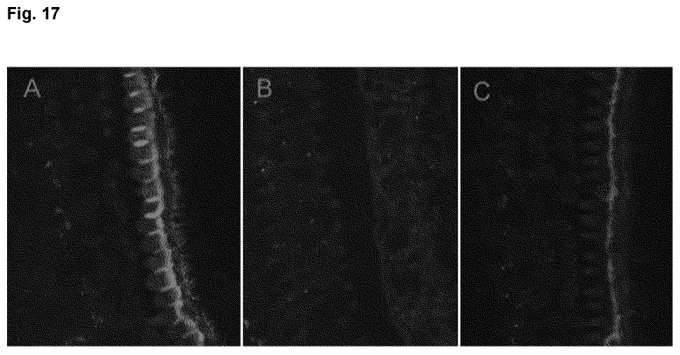

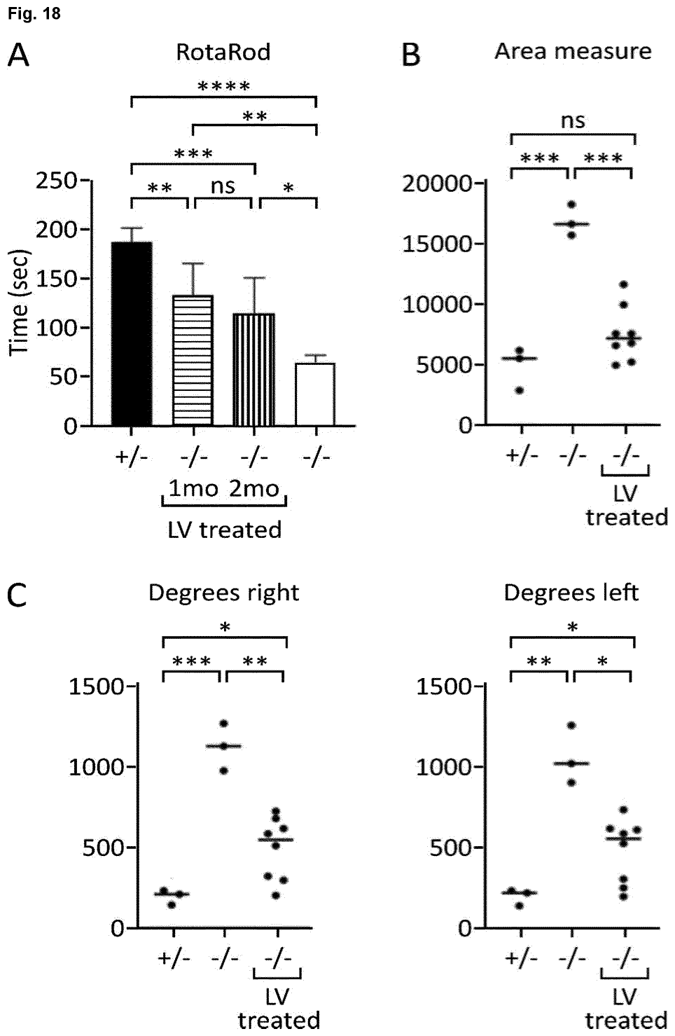

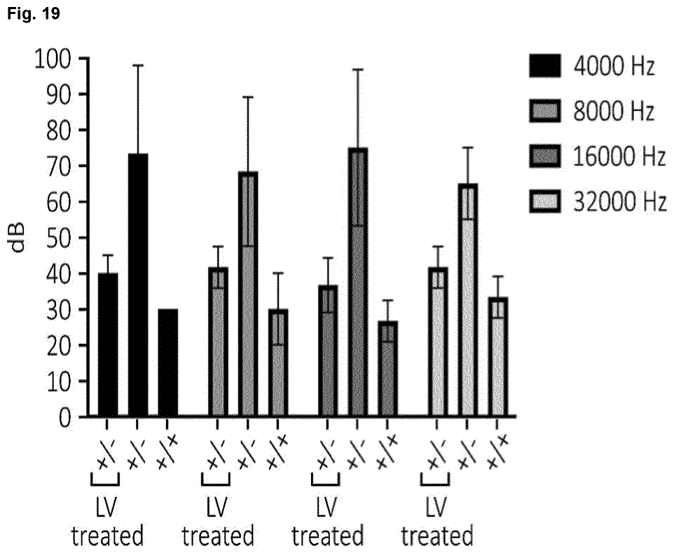

Functionally, lentiviral delivery of MYO7A led to increased survival of inner ear hair cells in the Shaker-1 mouse model, in which hearing loss is due to Myo7a knock-out (FIG. 17). Further demonstrating the gene therapy potential of LV particles, balance function was improved in homozygous Shaker-1 mice by LV delivery of MYO7A (FIG. 18). Auditory brainstem recordings (ABR) to determine the hearing level (e.g. threshold dB) at various sound frequencies demonstrated heterozygous Shaker-1 mice treated with the MYO7A-expressing LV to have vastly better hearing as compared to the untreated controls, indicating that the gene transfer inhibited the progressive hearing loss common to these mice (FIG. 19).

### REFERENCES

Blanc et al. (2020) rAAV-Mediated Cochlear Gene Therapy: Prospects and Challenges for Clinical Application. J. Clin Med. 9(2), 589.
Izumikawa et al. (2005) Auditory hair cell replacement and hearing improvement by Atoh1 gene therapy in deaf mammals. Nature Medicine 11(3), 271-276.
Tao et al. (2018) Delivery of Adeno-Associated Virus Vectors in Adult Mammalian Inner-Ear Cell Subtypes Without Auditory Dysfunction. Human Gene Therapy 29(4), 492-506.
Dong et al. (1996) Quantitative Analysis of the Packaging Capacity of Recombinant Adeno-Associated Virus. Human Gene Therapy 7, 2101-2112.
Milone and O'Doherty (2018) Clinical use of lentiviral vectors. Leukemia 32, 1529-1542.
Kumar et al. (2001) Systematic Determination of the Packaging Limit of Lentiviral Vectors. Human Gene Threapy 12, 1893-1905.
Schambach et al. (2013) Biosafety Features of Lentiviral Vectors. Human Gene Therapy 24, 132-142.
Zhang et al. (2001) A highly efficient and consistent method for harvesting large volumes of high-titre lentiviral vectors. Gene Therapy 8, 1745-1751.
Hashimoto et al. (2007) Lentiviral gene replacement therapy of retinas in a mouse model for Usher syndrome type 1B. Gene Therapy 14, 584-594.
Han et al. (1999) Transgene Expression in the Guinea Pig Cochlea Mediated by a Lentivirus-Derived Gene Transfer Vector. Human Gene Therapy 10, 1867-1873.
Pietola et al. (2009) HOX-GFP and WOX-GFP lentivirus vectors for inner ear gene transfer. Acta Oto-Laryngologica, 128, 613-620.
Yan Wei et al. (2013) Effect of lentiviruses carrying enhanced green fluorescent protein injected into the scala media through a cochleostomy in rats. American Journal of Otolaryngology-Head and Neck Medicine and Surgery 34, 301-307.
Pan et al. (2013) Lentivirus carrying the Atoh1 gene infects normal rat cochlea. Neural Regen Res. 8(17), 1551-1559.
Morgan et al. (2020) Gene therapy as a possible option to treat hereditary hearing loss. Medizinische Genetik.
ACMG Working Group on Update of Genetics Evaluation Guidelines for the Etiologic Diagnosis of Congenital Hearing Loss; for the Professional Practice and Guidelines Committee (2014) American College of Medical Genetics and Genomics guideline for the clinical evaluation and etiologic diagnosis of hearing loss. Genetics in Medicine 16, 347-355.
Cabanillas et al. (2018) Comprehensive genomic diagnosis of non-syndromic and syndromic hereditary loss in Spanish patients. BMC Medical Genomics 11, 58.
German Advisory Committee Blood (Arbeitskreis Blut), Subgroup ‘Assessment of Pathogens Transmissible by Blood’ (2016) Human Immunodeficiency Virus (HIV). Transfus Med Hemother. 43(3), 203-222.
Naldini et al., (1996) In vivo gene delivery and stable transduction of nondividing cells by a lentiviral vector. Science 272(5259). 263-267.
Zufferey et al. (1998) Self-Inactivating Lentivirus Vector for Safe and Efficient in Vivo Gene Delivery. J Virol 72(12), 9873-9880.
Iwamoto et al. (2010) A general chemical method to regulate protein stability in the mammalian centralnervous system. Chem Biol. 17(9), 981-988.
Lévy et al. (1999) Analysis of a conditional degradation signal in yeast and mammalian cells. Eur J Biochem. 259(1-2), 244-252.
Stankunas et al. (2003) Conditional protein alleles using knockin mice and a chemical inducer of dimerization. Mol Cell. 12(6), 1615-1624.
Catalanotto et al. (2016) MicroRNA in Control of Gene Expression: An Overview of Nuclear Functions. International journal of molecular sciences, 17(10), 1712.
Sage et al. (2005) Proliferation of functional hair cells in vivo in the absence of the retinoblastoma protein. Science 307(5712) 1056-1058.
Shaw and Cornetta (2014) Design and Potential of Non-Integrating Lentiviral Vectors. Biomedicines 2(1), 14-35.
Agnello et al. (1999) Hepatitis C virus and other flaviviridae viruses enter cells via a low density lipoprotein receptor. Proc Natl Acad Sci 96(22), 12766-12771.
Finkelshtein et al. (2013) LDL receptor and its family members serve as the cellular receptors for vesicular stomatitis virus. PNAS 110(18), 7306-7311.
Hosokawa et al. (2018) Immunohistochemical localization of megalin and cubilin in the human inner ear. Brain Res. 1701, 153-160.
Trobridge et al. (2010) Cocal-pseudotyped Lentiviral Vectors Resist Inactivation by Human Serum and Efficiently Transduce Primate Hematopoietic Repopulating Cells. Molecular Therapy 18(4), 725-733.
Scalise et al. (2018) The Human SLC1A5 (ASCT2) Amino Acid Transporter: FROM Function to Structure and Role in Cell Biology. Front Cell Dev Biol. 6(96).
Marin et al. (2003) N-Linked Glycosylation and Sequence Changes in a Critical Negative Control Region of the ASCT1 and ASCT2 Neutral Amino Acid Transporters Determine Their Retroviral Receptor Functions. Journal of Virology 77(5), 2936-2945.
Sandrin et al. (2002) Lentiviral vectors pseudotyped with a modified RD114 envelope glycoprotein show increased stability in sera and augmented transduction of primary lymphocytes and CD34+ cells derived from human and nonhuman primates. Blood 100(3), 823-32.
Girard-Gagnepain et al. (2014) Baboon envelope pseudotyped LVs outperform VSV-G-LVs for gene transfer into early-cytokine-stimulated and resting HSCs. Blood 124(8), 1221-1231.
Tijani et al. (2018) Lentivector Producer Cell Lines with Stably Expressed Vesiculovirus Envelopes. Mol Ther Methods Clin Dev. 10, 303-312.
Schambach et al. (2006) Equal potency of gammaretroviral and lentiviral SIN vectors for expression of O⁶-methylguanine-DNA methyltransferase in hematopoietic cells. Mol. Ther. 13(2), 391-400.
Meas et al. (2018) Reprogramming Glia Into Neurons in the Peripheral Auditory System as a Solution for Sensorineural Hearing Loss: Lessons From the Central Nervous System. Front Mol Neurosci. 11: 77.
Malgrange et al. (2002) Epithelial supporting cells can differentiate into outer hair cells and Deiters' cells in the cultured organ of Corti. Cellular and Molecular Life Sciences 59, 1744-1757.
Hendricks et al. (2008) Localized Cell and Drug Delivery for Auditory Prostheses. Hear Res. 242(1-2), 117-131.
Shibata et al. (2017) Intravenous rAAV2/9 injection for murine cochlear gene delivery. Sci. Rep. 7, 9609.

1. A method for treating sensorineural hearing loss in a subject in need thereof, comprising administering to the subject a composition comprising a 3^{rd} generation lentiviral vector pseudotyped with a viral envelope glycoprotein capable of binding to a receptor expressed in a cell of the inner ear, wherein said composition has a titer of at least 10⁷ TU/mL and is administered to the inner ear of the subject.

2. The method of claim 1, wherein the lentiviral vector is pseudotyped with a viral envelope glycoprotein capable of binding to a receptor selected from the group consisting of the LDL-receptor and LDL-R family members, the SLC1A5-receptor, the Pit1/2-receptor and the PIRYV-G-receptor.

3. The method of claim 1, wherein the lentiviral vector is pseudotyped with a viral envelope glycoprotein capable of binding the LDL-receptor, wherein the viral envelope glycoprotein is selected from the group comprising MARAV-G, COCV-G, VSV-G and VSV-G ts.

4. The method of claim 3, wherein the viral envelope glycoprotein is MARAV-G.

5. The method of claim 3, wherein the viral envelope glycoprotein is COCV-G.

6. The method of claim 3, wherein the viral envelope glycoprotein is VSV-G, such as wild-type VSV-G or VSV-G ts.

7. The method of claim 1, wherein the lentiviral vector comprises a cargo sequence comprising a nucleic acid selected from the group comprising a protein-coding gene, a miRNA, an shRNA, a lncRNA, or an sgRNA,
wherein expression of said cargo sequence in said cell of the inner ear is capable of reducing or eliminating at least a symptom of sensorineural hearing loss in the subject.

8. The method of claim 1, wherein the cargo sequence is a gene selected from the group comprising MYO7A, GJB2, OTOF, ATOH1, SLC26A4, TMC1, TMPRSS3, GIPC3, USH1C, RDX, CLDN14, WHRN, ESPN, ILDR1, SERPINB6, KCNQ4, POU4F3, SIX1, CCDC50, MIR96, POU3F4, EYA1, KCNQ1, GJB6, STRC, TECTA, GJB3, PRPS1, CDH23, GRXCR2, CABP2, GRXCR1, SYNE4, COL11A2, LRTOMT, BSND, PJVK, CIB2, CEACAM16, TMIE, NARS2, LHFPL5, MARVELD2, SLC22A4, KARS, SLC26A5, TPRN, ELMOD3, GPSM2, COCH, ESRRB, ADCY1, EPS8L2, CD164, OTOA, S1PR2, LOXHD1, TSPEAR, EPS8, CDC14A, MSRB3, DCDC2, PNPT1, TMEM132E, FAM65B, MYO3A, CLIC5, HGF, USH2A, TBC1D24, MET, PCDH15, PTPRQ, OTOGL, MYO6, TNC, TRIOBP, BDP1, MYO15A, DIAPH1, MYH14, DFNA5, WFS1, EYA4, COL11A2, MYH9, ACTG1, SLC17A8, GRHL2, DSPP, P2RX2, MYO1A, GFI1, TYMP, GFI1B, BDNF, GDNF, NT-3, IGF1, IGF2, CNTF, ARTN, NRTN, ATP7B, MRP1, MRP2 and MDR1.

9. The method of claim 1, wherein the lentiviral vector is derived from a virus selected from the group comprising HIV-1, HIV-2, SIV, FIV and EIAV, wherein, preferably, the lentiviral vector is derived from HIV-1.

10. The method of claim 1, wherein the lentiviral vector comprises
a) a 5′ LTR comprising a constitutively active heterologous promoter at the U3 position, a repeat region (R) and a U5 region,
b) a 5′ UTR comprising a primer binding site (PBS), a splice donor site (SD), a packaging signal (ψ), a Rev-responsive element, and, optionally, a splice acceptor (SA) site.
c) an internal enhancer/promoter region operably linked to a cargo sequence,
d) RNA processing elements optionally comprising a Woodchuck hepatitis virus posttranscriptional regulatory element (PRE), and
e) a 3′ LTR with a deleted U3 (SIN) region, a repeat region (R) and a U5 region.

11. The method of claim 1, wherein the cell of the inner ear is a cell selected from the group consisting of cells of the organ of Corti including supporting cells as well as inner and outer hair cells, spiral ganglion neurons and glial cells, cells of the stria vascularis and spiral ligament, lateral wall fibrocytes, type I and II vestibular hair cells, vestibular supporting cells and vestibular ganglion neurons.

12. The method of claim 1, wherein the lentiviral vector titer is concentrated using ultracentifugation or other purification methods, incl. microfiltration, ultrafiltration, chromatography, density gradient ultracentrifugation, tangential flow microfiltration, and precipitation.

13. The method of claim 1, wherein the composition is administered to the subject by a method selected from the group comprising a cochlea implant route, round window injection, oval window injection, canalostomy, cochleostomy and injection into the endolymphatic sac.

14. The method of claim 1, wherein the lentiviral vector is a non-integrating lentiviral vector.

15. (canceled)
